SEQUENCE LISTING (85 sequences)

SEQ ID NO: 1 - DNA, 27 bp, Mus musculus
  CDS (1)..(27)
tgttcctacggcttatatagctcaggt

SEQ ID NO: 2 - protein, 9 aa, Mus musculus
CSYGLYSSG

SEQ ID NO: 3 - DNA, 27 bp, Mus musculus
  CDS (1)..(27)
tgttcctacggcctatatagctcaggt

SEQ ID NO: 4 - protein, 9 aa, Mus musculus
CSYGLYSSG

SEQ ID NO: 5 - DNA, 27 bp, Mus musculus
  CDS (1)..(27)
tgttcctacggtctatatagctcaggt

SEQ ID NO: 6 - protein, 9 aa, Mus musculus
CSYGLYSSG

SEQ ID NO: 7 - DNA, 27 bp, Mus musculus
  CDS (1)..(27)
tgttcctacggactatatagctcaggt

SEQ ID NO: 8 - protein, 9 aa, Mus musculus
CSYGLYSSG

SEQ ID NO: 9 - DNA, 27 bp, Mus musculus
  CDS (1)..(27)
tgttcctacggcctctatagctcaggt

SEQ ID NO: 10 - protein, 9 aa, Mus musculus
CSYGLYSSG

SEQ ID NO: 11 - DNA, 27 bp, Mus musculus
  CDS (1)..(27)
tgttcctacggcttgtatagctcaggt

SEQ ID NO: 12 - protein, 9 aa, Mus musculus
CSYGLYSSG

SEQ ID NO: 13 - protein, 13 aa, Mus musculus
LMERGGGIRATDK

SEQ ID NO: 14 - protein, 14 aa, Mus musculus
LMERDIGGIRATDK

SEQ ID NO: 15 - protein, 14 aa, Mus musculus
LMERGIGGIRATDK

SEQ ID NO: 16 - protein, 14 aa, Mus musculus
LMERAIGGIRATDK

SEQ ID NO: 17 - protein, 14 aa, Mus musculus
LMGRHIGGIRATDK

SEQ ID NO: 18 - protein, 14 aa, Mus musculus
LMERGNGGIRATDK

SEQ ID NO: 19 - protein, 14 aa, Mus musculus
LMERGSGGIRATDK

SEQ ID NO: 20 - protein, 14 aa, Mus musculus
LMERNIGGIRATDK

SEQ ID NO: 21 - protein, 14 aa, Mus musculus
LMERVIGGIRATDK

SEQ ID NO: 22 - protein, 14 aa, Mus musculus
LMERGIGGIRATDK

SEQ ID NO: 23 - protein, 16 aa, Mus musculus
LMERVAYIGGIRAPDK

SEQ ID NO: 24 - protein, 16 aa, Mus musculus
LMERVAYPPIGGIRAD

SEQ ID NO: 25 - protein, 12 aa, Mus musculus
LMETGGIRATDK

SEQ ID NO: 26 - protein, 10 aa, Mus musculus
LMVGGRTTDK

SEQ ID NO: 27 - protein, 13 aa, Mus musculus
LMENIGGIRATDK

SEQ ID NO: 28 - DNA, 27 bp, Mus musculus
  CDS (1)..(27)
tgttcctacggtctatatagctcaggt

SEQ ID NO: 29 - protein, 9 aa, Mus musculus
CSYGLYSSG

SEQ ID NO: 30 - DNA, 27 bp, Mus musculus
  CDS (1)..(27)
tgttcctacggcctttatagctcaggt

SEQ ID NO: 31 - protein, 9 aa, Mus musculus
CSYGLYSSG

SEQ ID NO: 32 - DNA, 27 bp, Mus musculus
  CDS (1)..(27)
tgttcctacggcttatatagctcaggt

SEQ ID NO: 33 - protein, 9 aa, Mus musculus
CSYGLYSSG

SEQ ID NO: 34 - DNA, 24 bp, Mus musculus
  CDS (1)..(24)
tgttcctacggctatagctcaggt

SEQ ID NO: 35 - protein, 8 aa, Mus musculus
CSYGYSSG

SEQ ID NO: 36 - DNA, 27 bp, Mus musculus
  CDS (1)..(27)
tgttcctacggcccatatagctcaggt

SEQ ID NO: 37 - protein, 9 aa, Mus musculus
CSYGPYSSG

SEQ ID NO: 38 - DNA, 18 bp, Mus musculus
  CDS (1)..(18)
tgttccgggagctcaggt

SEQ ID NO: 39 - protein, 6 aa, Mus musculus
CSGSSG

SEQ ID NO: 40 - DNA, 27 bp, Mus musculus
  CDS (1)..(27)
tgttcctacggcggatatagctcaggt

SEQ ID NO: 41 - protein, 9 aa, Mus musculus
CSYGGYSSG

SEQ ID NO: 42 - DNA, 27 bp, Mus musculus
  CDS (1)..(27)
tgttcctacggcaagaggagctcaggt

SEQ ID NO: 43 - protein, 9 aa, Mus musculus
CSYGKRSSG

SEQ ID NO: 44 - DNA, 21 bp, Mus musculus
  CDS (1)..(21)
tgttcctacggcagctcaggt

SEQ ID NO: 45 - protein, 7 aa, Mus musculus
CSYGSSG

SEQ ID NO: 46 - DNA, 27 bp, Mus musculus
  CDS (1)..(27)
tgttcctacggcgaatatagctcaggt

SEQ ID NO: 47 - protein, 9 aa, Mus musculus
CSYGEYSSG

SEQ ID NO: 48 - DNA, 27 bp, Mus musculus
  CDS (1)..(27)
tgttcctacggggtatatagctcaggt

SEQ ID NO: 49 - protein, 9 aa, Mus musculus
CSYGVYSSG

SEQ ID NO: 50 - DNA, 24 bp, Mus musculus
  CDS (1)..(24)
tgttcctacgggaatagctcaggt

SEQ ID NO: 51 - protein, 8 aa, Mus musculus
CSYGNSSG

SEQ ID NO: 52 - DNA, 24 bp, Mus musculus
  CDS (1)..(24)
tgttcctacggctacggctcaggt

SEQ ID NO: 53 - protein, 8 aa, Mus musculus
CSYGYGSG

SEQ ID NO: 54 - DNA, 24 bp, Mus musculus
  CDS (1)..(24)
tgttcctacggggatagctcaggt

SEQ ID NO: 55 - protein, 8 aa, Mus musculus
CSYGDSSG

SEQ ID NO: 56 - DNA, 27 bp, Mus musculus
  CDS (1)..(27)
tgttcctacggcgcatatagctcaggt

SEQ ID NO: 57 - protein, 9 aa, Mus musculus
CSYGAYSSG

SEQ ID NO: 58 - DNA, 27 bp, Mus musculus
  CDS (1)..(27)
tgttcctacggctcctatagctcaggt

SEQ ID NO: 59 - protein, 9 aa, Mus musculus
CSYGSYSSG

SEQ ID NO: 60 - DNA, 42 bp, Mus musculus
  CDS (1)..(42)
ctcatggagcgcggtatcggagggatacgagctaccgacaaa

SEQ ID NO: 61 - protein, 14 aa, Mus musculus
LMERGIGGIRATDK

SEQ ID NO: 62 - DNA, 45 bp, Mus musculus
  CDS (1)..(45)
ctcatggagcgggggatcggagggatacaggctcgggccgacaaa

SEQ ID NO: 63 - protein, 15 aa, Mus musculus
LMERGIGGIQARADK

SEQ ID NO: 64 - DNA, 42 bp, Mus musculus
  CDS (1)..(42)
ctcatggagcgcgaaggcggagggatacgtgctaccgacaaa

SEQ ID NO: 65 - protein, 14 aa, Mus musculus
LMEREGGGIRATDK

SEQ ID NO: 66 - DNA, 54 bp, Mus musculus
  CDS (1)..(54)
ctcatggagcgcgaatggtggcaccgatcgatcggagggatagctaccgacaaa

SEQ ID NO: 67 - protein, 18 aa, Mus musculus
LMEREWWHRSIGGIATDK

SEQ ID NO: 68 - DNA, 54 bp, Mus musculus
  CDS (1)..(54)
ctcatggagcgcctgcgcccgggtatcggagggtacccctctgctaccgacaaa

SEQ ID NO: 69 - protein, 18 aa, Mus musculus
LMERLRPGIGGYPSATDK

SEQ ID NO: 70 - DNA, 45 bp, Mus musculus
  CDS (1)..(45)
ctcatggagcgcgagacatatcggagggatccagctaccgacaaa

SEQ ID NO: 71 - protein, 15 aa, Mus musculus
LMERETYRRDPATDK

SEQ ID NO: 72 - DNA, 45 bp, Mus musculus
  CDS (1)..(45)
ctcatggagcgcgctgcggcatatcggagggataaaaccgacaaa

SEQ ID NO: 73 - protein, 15 aa, Mus musculus
LMERAAAYRRDKTDK

SEQ ID NO: 74 - DNA, 39 bp, Mus musculus
  CDS (1)..(39)
ctcatggagcgtggcatatgctggaggggtaccgacaaa

SEQ ID NO: 75 - protein, 13 aa, Mus musculus
LMERGICWRGTDK

SEQ ID NO: 76 - DNA, 42 bp, Mus musculus
  CDS (1)..(42)
ctcatggagcgcggtatatgtcggagggatgctaccgacaaa

SEQ ID NO: 77 - protein, 14 aa, Mus musculus
LMERGICRRDATDK

SEQ ID NO: 78 - DNA, 33 bp, Mus musculus
  CDS (1)..(33)
ctcatggagcgcgttctccgggctaccgacaaa

SEQ ID NO: 79 - protein, 11 aa, Mus musculus
LMERVLRATDK

SEQ ID NO: 80 - DNA, 39 bp, Mus musculus
  CDS (1)..(39)
ctcatggagcgcgcccggagggatacgcggaccgacaaa

SEQ ID NO: 81 - protein, 13 aa, Mus musculus
LMERARRDTRTDK

SEQ ID NO: 82 - DNA, 42 bp, Mus musculus
  CDS (1)..(42)
ctcatggagctcgggccatcagagggatacgagtccgacaaa

SEQ ID NO: 83 - protein, 14 aa, Mus musculus
LMELGPSEGYESDK

SEQ ID NO: 84 - DNA, 42 bp, Mus musculus
  CDS (1)..(42)
ctcatggagcggctatcggagggatacgagctgaccgacaaa

SEQ ID NO: 85 - protein, 14 aa, Mus musculus
LMERLSEGYELTDK

(19) United States
(12) Patent Application Publication
(10) Pub. No.: US 2008/0248025 A1
(43) Pub. Date: Oct. 9, 2008

(54) Gamma Delta T Cells and Methods of Treatment of Interleukin-17 Related Conditions

(75) Inventors: Christina Roark, Englewood, CO (US); Rebecca L. O'Brien, Denver, CO (US); Willi K. Born, Denver, CO (US)

Correspondence Address: SHERIDAN ROSS PC, 1560 BROADWAY, SUITE 1200, DENVER, CO, 80202, US

(73) Assignee: NATIONAL JEWISH MEDICAL AND RESEARCH CENTER, Denver, CO (US)

(21) Appl. No.: 12/053,310

(22) Filed: Mar. 21, 2008

Related U.S. Application Data
(60) Provisional application No. 60/908,278, filed on Mar. 27, 2007.

(51) Int. Cl. A61K 39/395; A61K 38/17; C12Q 1/02; A61P 35/00; A61P 37/00

(52) U.S. Cl. 424/130.1; 514/12; 435/29

(57) ABSTRACT

This invention generally relates to methods to treat conditions and diseases associated with interleukin-17 (IL-17) production. The invention also relates to methods of inhibiting γδ T cells, and particularly, a subset of γδ T cells that produce IL-17.

### CROSS-REFERENCE TO RELATED APPLICATIONS

This application claims the benefit of priority under 35 U.S.C. § 119(e) of U.S. Provisional Application No. 60/908,278, filed Mar. 27, 2007, the entire disclosure of which is incorporated herein by reference for all purposes.

### FIELD OF THE INVENTION

This invention generally relates to methods to treat conditions and diseases associated with interleukin-17 (IL-17) production. The invention also relates to methods of inhibiting γδ T cells, and particularly, a subset of γδ T cells that produce IL-17.

### BACKGROUND OF THE INVENTION

Autoimmune disorders are conditions caused by an immune response against the body's own tissues. Autoimmune disorders result in destruction of one or more types of body tissues, abnormal growth of an organ, or changes in organ function. The disorder may affect only one organ or tissue type or may affect multiple organs and tissues. For example, rheumatoid arthritis (RA) is a chronic autoimmune disease that causes inflammation of the joints and surrounding tissues, but can also affect other organs. Autoimmune diseases and other diseases involving chronic inflammation affect many people worldwide and are the topic of a large volume of research. Fortunately, multiple animal models of autoimmune diseases exist that provide valuable tools for identifying therapeutic strategies for the treatment of these diseases.

Collagen-induced arthritis (CIA) is a murine model of chronic inflammation that shares many hallmarks with rheumatoid arthritis (RA) (reviewed in¹). For example, there is a strong association with the MHC Class II allele HLA-DR4 (DRB1*0401) in humans and IA^{q} in mice^{2,3} and both class II molecules bind the same immunodominant collagen type II (CII) peptide⁴. In addition, anti-collagen antibodies play a critical role in the development of CIA (reviewed in¹) and complement-fixing IgG2a has been shown to dominate the anti-collagen response and be essential for pathogenesis⁵. Finally, αβ T cells have been shown to be essential in CIA⁶.

There is also evidence that γδ T cells play a role in CIA^{7,8}. γδ T cells are resident in the synovium of mice and their proportion in the joints rises dramatically when mice develop CIA^{7,8}. Additionally, γδ T cells are increased in the peripheral blood and synovium of patients with RA⁹⁻¹¹. However, studies in mice genetically deficient for T cells have shown that γδ T cells are neither necessary nor sufficient for the development of CIA⁶. Yet, when mice were temporarily depleted of γδ T cells, an effect on disease was noted. Depleting mice of γδ T cells prior to immunization with CII significantly delayed the onset of arthritis and severity. In contrast, antibody administered 40 days after the immunization resulted in rapid and severe exacerbation of CIA⁷.

### SUMMARY OF THE INVENTION

One aspect of the invention relates to a method to reduce the severity or incidence of a disease or condition associated with the production of interleukin-17 (IL-17) comprising deleting, inactivating or inhibiting γδ T cells in an individual who has or is at risk of developing the disease.

In some embodiments, the disease is an autoimmune disease such as rheumatoid arthritis, systemic lupus erythematosus, or multiple sclerosis.

In some embodiments, the disease is a cancer associated with the production of IL-17, such as a cancer of a mucosal tissue or organ. In certain embodiments, the cancer is selected from melanomas, squamous cell carcinomas, breast cancers, head and neck carcinomas, thyroid carcinomas, soft tissue sarcomas, bone sarcomas, testicular cancers, prostatic cancers, pancreatic cancers, ovarian cancers, uterine cancers, cervical cancers, bladder cancers, skin cancers, brain cancers, angiosarcomas, hemangiosarcomas, mast cell tumors, primary hepatic cancers, lung cancers (including non-small cell lung carcinomas), pancreatic cancers, gastrointestinal cancers (including colorectal cancers), renal cell carcinomas, hematopoietic neoplasias and metastatic cancers thereof. In some embodiments, the cancer is uterine cancer or colorectal cancer.

In some embodiments, the disease is an inflammatory condition associated with the production of IL-17, such as an inflammatory condition of a mucosal organ or tissue. In certain embodiments, the inflammatory condition is not a bacterial or mycobacterial infection.

In some embodiments, the method comprises selectively deleting, inactivating or inhibiting γδ T cells that produce IL-17 in the individual. In some embodiments, the γδ T cells in the individual produce IL-17 and express activation markers such as CD44.

In some embodiments, the γδ T cells have reduced expression of CD62L or CD45RB, as compared to γδ T cells in an individual that does not have the autoimmune disease.

In some embodiments, the method comprises deleting, inactivating or inhibiting a population of γδ T cells in the individual that produce IL-17 and have a T cell receptor comprised of the same Vγ and Vδ combination.

In some embodiments, the method comprises, inactivating or inhibiting a population of γδ T cells in the individual that produce IL-17 and have a T cell receptor with a highly conserved amino acid motif in the CDR3 region of the TCR-δ chain.

In some embodiments, the method comprises deleting, inactivating or inhibiting a population of γδ T cells in the individual that produce IL-17 and have a T cell receptor with a highly conserved amino acid motif in the CDR3 region of the TCR-γ chain.

In some embodiments, the method comprises deleting, inactivating or inhibiting γδ T cells having a T cell receptor comprising Vγ4 or the human equivalent thereof.

In some embodiments, the method comprises deleting, inactivating or inhibiting γδ T cells having a T cell receptor comprising Vγ4 or the human equivalent thereof, and comprising Vδ4 or the human equivalent thereof.

In some embodiments, the γδ T cells are deleted, inactivated or inhibited by selective leukophoresis.

In some embodiments, the γδ T cells are deleted, inactivated or inhibited by administration of an agent that selectively targets γδ T cells.

In some embodiments, the γδ T cells are deleted, inactivated or inhibited by administration of an agent that selectively targets γδ T cells having a specified Vγ and Vδ combination.

In some embodiments, the γδ T cells are deleted, inactivated or inhibited by administration of an agent that selectively targets γδ T cells expressing TCR-Vγ4, or the human equivalent thereof.

In some embodiments, the γδ T cells are deleted, inactivated or inhibited by administration of an agent that selectively targets γδ T cells expressing TCR-Vγ4/Vδ4, or the human equivalent thereof.

In some embodiments, the agent is an antibody or antigen-binding fragment thereof.

In some embodiments, the agent is a soluble γδ T cell receptor identical or equivalent to that expressed by the γδ T cells to be deleted, inactivated or inhibited.

Another aspect of the invention relates to a method to reduce the severity or incidence of rheumatoid arthritis in an individual, comprising deleting, inactivating or inhibiting γδ T cells that produce interleukin-17 (IL-17) in the individual.

In some embodiments, the method comprises selectively deleting, inactivating or inhibiting γδ T cells in the joints of the individual.

Another aspect of the invention relates to a method to reduce the severity or incidence of systemic lupus erythematosus in an individual, comprising deleting, inactivating or inhibiting γδ T cells that produce interleukin-17 (IL-17) in the individual.

Another aspect of the invention relates to a method to reduce the severity or incidence of multiple sclerosis in an individual, comprising deleting, inactivating or inhibiting γδ T cells that produce interleukin-17 (IL-17) in the individual.

Another aspect of the invention relates to a method to identify an agent useful for the treatment of a disease or condition associated with the production of interleukin-17 (IL-17) by contacting γδ T cells that produce IL-17 with a putative agent, wherein the γδ T cells were obtained or derived from a patient with a disease or condition associated with the production of IL-17, and selecting a putative agent that deletes or inactivates the γδ T cells as an agent for the treatment of the disease or condition.

Another aspect of the invention relates to a method to identify an agent useful for the treatment of a disease or condition associated with the production of interleukin-17 (IL-17) comprising contacting γδ T cells that produce IL-17 and that express TCR-Vδ4, or the human equivalent thereof, with a putative agent, and selecting a putative agent that deletes or inactivates the γδ T cells of (a) as an agent for the treatment of the disease or condition.

In some embodiments, the step of selecting comprises selecting a putative agent that inhibits the production of IL-17 by the γδ T cells.

In some embodiments, the γδ T cells express TCR-Vγ4/Vδ4, or the human equivalent thereof.

In some embodiments, the disease is an autoimmune disease.

In some embodiments, the autoimmune disease is rheumatoid arthritis.

In some embodiments, the autoimmune disease is systemic lupus erythematosus.

In some embodiments, the disease is a cancer.

In some embodiments, the condition is an inflammatory condition.

In some embodiments, the condition is an inflammatory condition associated with a mucosal tissue or organ.

### BRIEF DESCRIPTION OF THE DRAWINGS OF THE INVENTION

FIG. 1. The total numbers of γδ T cells (a), Vγ1⁺ cells, and Vγ4⁺ cells (b) obtained from the lymph nodes of mice that had received collagen/CFA injections on days 0 and 21 (black arrows). On the indicated days following the initial injection, the draining lymph nodes (inguinal, brachial and popliteal) were removed and cells were stained for γδ T cell subsets. Using FACs analysis, the total number of γδ cells and individual subsets were calculated. Each time point represents the average+SEM for at least 8 different mice. (c) On designated days after collagen/CFA injections (black arrows), γδ T cells were isolated and stained for Vγ1 and Vγ4 expression and for levels of CD62L, CD44, or CD45RB. The mean percentage+SEM of cells having an “activated” phenotype (CD62L low, CD44 high, CD45RB low) is shown.

FIG. 2. (a) Clinical disease activity in mice with collagen-induced arthritis. Mice were immunized with collagen/CFA on days 0 and 21. On day 17, mice were given either anti-Vγ4 antibody (black triangle, n=30) or hamster IgG (hIgG) (black square, n=26) intravenously. (b) In a separate experiment, mice were treated with anti-Vγ1 antibody (black triangle, n=30) or hamster IgG (black square, n=25). Clinical disease was assessed three times a week, starting on day 21. Values represent the mean ±SEM of 2 separate experiments (maximum score=16). **p<0.01, ***p<0.001. Statistical significance was determined using the Mann-Whitney test. (c) Incidence of disease for each of the groups as described in (a & b). The incidence of disease for each hIgG treated group in 2 experiments was pooled and is shown as one group. (d) Anti-collagen antibody levels in mice with CIA that were depleted with an anti-Vγ4 antibody, as compared to hIgG-injected control animals. Mean ±SEM is shown for total IgG, IgG1, and IgG2a antibodies. Mice were treated on day 17 with hIgG (black square, n=26) or a specific anti-Vγ subset antibody (black triangle, n=30). *p<0.05, ***p<0.001. Data represent the mean ±SEM of 2 separate experiments. (e) Same as in (d) except mice were depleted with an anti-Vγ1 antibody and compared to hIgG. Again, mean ±SEM is shown for 2 separate experiments.

FIG. 3. Intracellular cytokine staining of T cells from the draining lymph nodes on day 26. (a) The percentages of CD4⁺, γδ⁺, Vγ1⁺, or Vγ4⁺ T cells that can produce IL-17 are shown, first gating on cells that stained with CD3. The percentage of Vγ1⁺ and Vγ4⁺ cells was then visualized by next gating on cells that stained with a pan-γδ reactive MA\tilde{b} (b) The total number of Vγ1⁺, Vγ4⁺, or CD4⁺ cells stimulated to produce IL-17, IFNγ, IL-2, or TNFα as determined by intracellular cytokine staining. The total number was calculated based on the percentage that stained in (a). (c) The percentage of Vγ4⁺ cells in the joints of naïve mice versus CIA mice on day 26 is depicted on the left. The percentage of γδ⁺/Vγ4⁺ cells that can produce IL-17 is shown on the right.

FIG. 4. Vδ usage by Vδ4⁺ cells in CIA animals. Lymph nodes were analyzed by flow cytometry as for FIG. 3. Cells were triple stained for γδ TCR, Vγ4, and either Vδ4 (a), Vδ5 (b), or Vδ6.3 (data not shown) and the percentage of each V delta subset determined. Then, each Vδ-defined subset (circled population) was examined intracellularly for IL-17 production. The majority of the IL-17-producing Vδ4⁺ cells co-expressed Vδ4. Vγ4/Vδ6.3⁺ cells represented less than 0.5% of the Vγ4⁺ population, and did not produce IL-17 (not shown).

FIG. 5. Vγ4 and Vδ4 sequences from CIA-elicited γδ T cells. (a) In the CIA-elicited cells, 37/42 (88%) of the Vγ4⁺ clones encoded a leucine between the V and the J, and four of the six possible codons were used. In addition, when the codon “cta” was used to form leucine, different N/P nucleotides were also found flanking it. (b) The Vδ4 sequences from CIA-elicited cells revealed a striking length conservation (5-6 amino acids between V and J), a single Dδ2 reading frame, and the conservation of the two arginines, one at the end of the Vδ4 gene and one at the end of the Dδ2 gene. Both arginines were encoded by multiple codons as well.

FIG. 6. (a) γδ T cells are present in the normal joint and increased in diseased joints. DBA/1 lac J mice were injected with collagen/CFA and diseased joints taken on day 35. Joints were stained and compared with joints taken from mice that had not received any injections. Only diseased joints from collagen injected mice were analyzed. (b) Intracellular cytokine staining on day 26 for IL-17 in cells from the joints of collagen/CFA injected mice.

FIG. 7. Vγ4 sequences in the lymph nodes of naive DBA/1 mice.

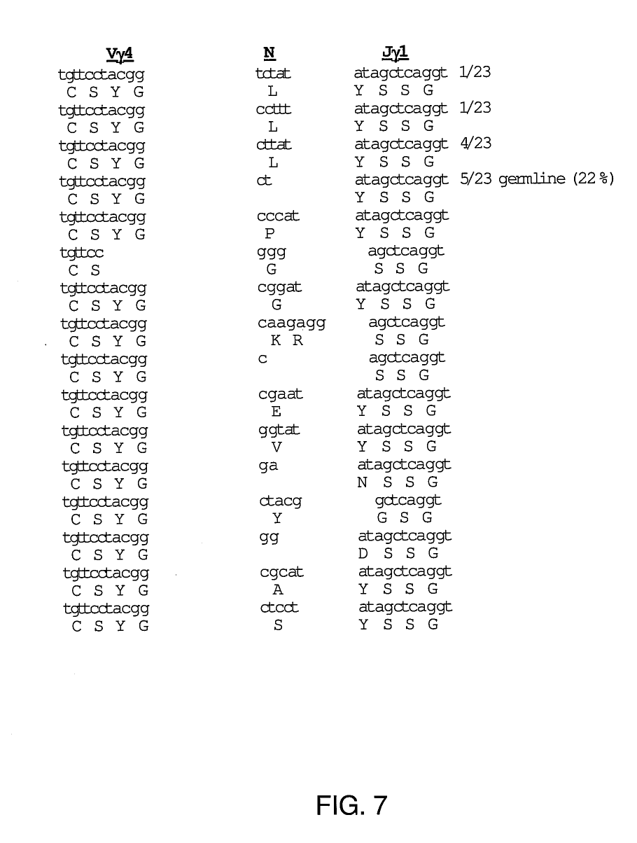

FIG. 8. Vδ4 sequences in the lymph nodes of naive DBA/1 mice.

### DESCRIPTION OF THE INVENTION

The invention generally relates to a method to treat conditions and diseases associated with interleukin-17 (IL-17) production, including, but not limited to, autoimmune diseases, cancers, and inflammatory diseases or conditions of the mucosal organs and tissues. In one embodiment, the invention relates to a method to treat rheumatoid arthritis, and particularly decrease the severity and incidence of rheumatoid arthritis, as well as related diseases, by inhibiting γδ T cells, and particularly, a subset of γδ T cells that produce interleukin-17 (IL-17).

In this experiments described herein, the inventors demonstrate an antigen-driven oligoclonal response by the Vγ4/Vδ4⁺γδ T cell subset. These cells are a potent source of IL-17 in the lymph nodes and joints of CIA mice and contribute to disease development. Therefore, without being bound by theory, the inventors propose a method to reduce chronic inflammation in diseases such as CIA by preventing or eliminating the response of certain subsets of γδ T cells. Accordingly, the invention also relates to therapies that target this small population of cells.

Previous studies have demonstrated that the two main peripheral γδ T cell subsets^{12,13}, Vγ1 and Vγ4, have different functional roles in various disease models (reviewed in¹⁴). In the CIA model, the inventors discovered that while both Vγ1⁺ and Vγ4⁺ cells increased, only the Vγ4⁺ cells were activated, as measured by surface marker expression. Depletion of Vγ4⁺ cells during CIA resulted in less severe disease indicating a pathogenic role for these cells. Because the proinflammatory cytokine, IL-17, has been shown to play an important pathogenic role in autoimmune diseases such as experimental allergic encephalomyelitis (EAE) and CIA (reviewed in¹⁵), the inventors also examined whether γδ T cell subsets could produce IL-17. The inventors discovered that the vast majority of the responding Vγ4⁺ cells produced IL-17 and co-expressed Vδ4. Sequence analysis revealed limited γ and δ junctional regions, indicating that these cells were antigen-selected.

The inventors' results indicate that Vγ4⁺, but not Vγ1⁺ cells, are pathogenic in CIA. First, although Vγ1⁺ and Vγ4⁺ cells both increased during CIA after the second injection, the Vγ4⁺ cells increased more rapidly, and to a greater extent, than the Vγ1⁺ subset. As well, many of the Vγ4⁺ γδ T cells expressed markers of activation following the collagen/CFA injections, while the Vγ1⁺ cells appeared unresponsive. Second, depletion of the Vγ4⁺ cells before the second collagen/CFA injection led to a decrease in the severity and incidence of CIA, which was not seen following depletion of Vγ1⁺ cells. Finally, only the Vγ4⁺ subset produced IL-17, which is associated with inflammatory damage in CIA. Since surface markers of activation have not been studied extensively on γδ T cells, it is possible that characteristics of activation for Vγ1⁺ cells are different. Moreover, removing Vγ1⁺ cells before induction of CIA or at another time point of the disease process might uncover a role for this subset as well.

While the function of γδ T cells often segregates with their Vγ chain usage, recent studies have demonstrated an important role for the TCRδ chain in ligand recognition. Shin et al. found that γδ T cell clone, denoted G8, recognized its ligand, T22^{b}, almost exclusively through the Dδ2 portion of the δ chain. Using a T22^{b}-tetramer to identify T22^{b}-specific γδ T cells, they found that a particular motif in the δ chain CDR3 was sufficient to confer T22^{b} binding²¹. This motif was generated by the use of Dδ2 in only one of three possible reading frames [encoding (S)EGYE], flanked by two other conserved residues, a preceding tryptophan encoded by either the 3′ end of Vδ6.3 or by Dδ1, and a following leucine encoded by N or P-nucleotide additions. A variety of CDR3δ lengths were permitted among T22^{b}-binding γδ TCRs, and the required motif could be generated almost entirely from germline-encoded components.

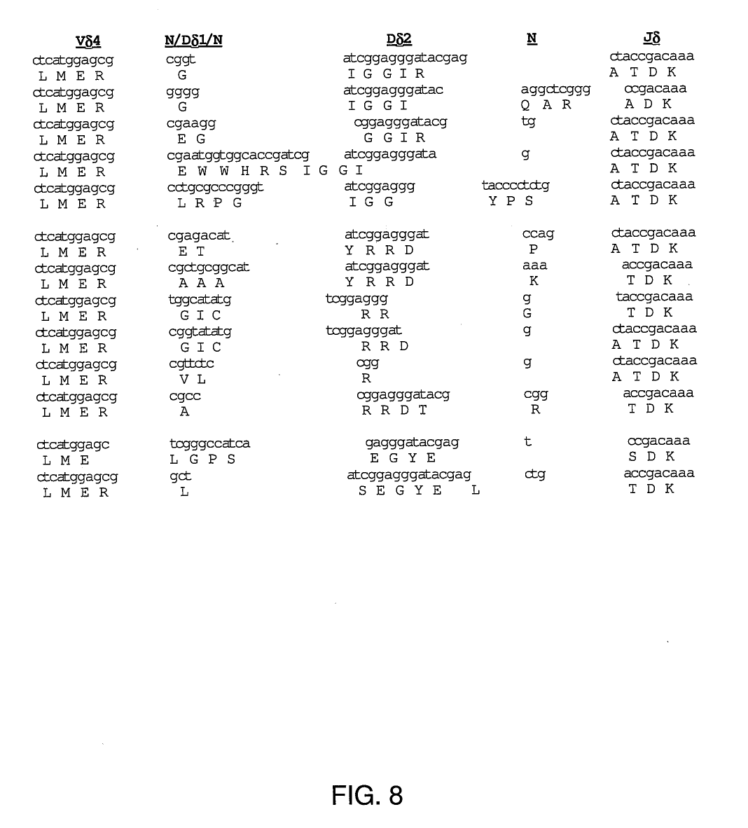

The inventors' findings indicate that γδ T cells bearing particular TCRs are preferentially expanded by an antigen present during CIA. However, the requirements for binding this putative antigen appear to include elements of both the γ and δ chains, because activated cells expressing the Vγ4/Vδ4 combination predominated. The inventors also found a recurrent motif in the CDR3 regions of the TCR-δ chain, including a single reading frame for Dδ2 among all CIA-elicited Vδ4s [(I) GGIR] (30/30 clones). Although the (I) GGIR reading frame is normally somewhat more common than the (S)EGYE reading frame, only 5/13 clones derived from naïve mice used the (I) GGIR reading frame (FIG. 8). The Dδ2 was preceded by an arginine in 27/30 clones, encoded by either Vδ4 or N/P nucleotides, which may explain the preference for this Vδ. Also, a second arginine, encoded by the 3′ end of Dδ2, was found in all 30 CIA-elicited Vδ4 clones, compared to only 4/13 naïve clones. Unlike the T22^{b}-reactive δ-chains, the lengths of the CIA-elicited δ-chain CDR3s also seemed to be restricted, ranging between 5-6 amino acids between V and J in 23/30 clones.

The CDR3 of the CIA-elicited Vγ4s was also very limited. 37/42 clones contained only a single amino acid, leucine, between V and J, and four of the six possible leucine codons were found, consistent with the selective expansion of Vγ4/Vδ4⁺ cells bearing a particular motif in the γ-CDR3 as well. This contrasts markedly with the findings for T22-binding γδTCRs, in which the δ chain appeared to be uninvolved in ligand interaction²¹. Indeed, the γδ TCR restrictions associated with the CIA-selected γδ T cells are reminiscent of those common for γδ TCRs specific for a given ligand. Therefore, γδ T cells in the CIA model appear to be selected in a manner different from the T22^{b}-binding cells, and more akin to the selection of αβ T cells. Thus, the question of whether γδ TCR ligand recognition differs fundamentally from that of αβ TCRs remains open.

Most of the molecules identified so far as ligands for γδ TCRs, including T22^{b}, appear to be host-encoded molecules whose expression is induced by inflammation or stress (reviewed in²³). However, the observed expansion of Vγ4/Vδ4⁺ cells in the inventors' model of CIA could be due to a response to the CFA, which is used in the immunizations. Therefore, the inventors have examined mice immunized with PBS/CFA, which does not cause CIA in DBA/1 mice. Similar to CII-injected mice, the total number of γδ, Vγ1⁺ and Vγ4⁺ T cells increased after each PBS/CFA injection and moreover, the Vγ4⁺ subset showed the same “activated” phenotype (high CD44, low CD62L, and low CD45RB expression) as before (data not shown). However, the timing of the response was different. Despite similar initial responses, the maximal response measured by the percentage of “activated” Vδ4⁺ cells after the second PBS/CFA immunization was delayed, peaking at 6 days versus 4 days in CIA mice (data not shown), perhaps indicating that different stimuli are triggered in CIA than by PBS/CFA treatment alone. Hybridomas are now being produced to further investigate whether Vγ4/Vδ4⁺ cells respond to collagen, or another host-derived molecule that is induced by the immunization.

Opposing roles for Vγ1⁺ and Vδ4⁺ cells in various disease models have been previously noted. For example, Vγ4⁺ cells suppress allergic airway hyperresponsiveness (AHR)²⁴ while Vγ1⁺ cells enhance AHR²⁵. In addition, Vδ4⁺ cells promote myocarditis in a coxsackievirus B3 model, whereas Vγ1⁺ cells are protective²⁶. This difference was attributed to skewing of the Th1/Th2 αβ T cell response by the γδ T cells. Interestingly, when using the BALB/c mouse in an effort to look at IL-4 producing CD4⁺ cells, infection with a strain of coxsackievirus B3 that promotes myocarditis resulted in an expansion of Vγ4/Vδ4⁺ cells²⁷. Intracellular cytokine staining of these Vγ4/Vδ4⁺ cells revealed that a large proportion (50%) produced IFNγ (IL-17 was not measured). In the inventors' model of CIA, only 4% of the Vγ4/Vδ4⁺ cells produced IFNγ (data not shown). These results indicate Vγ4/Vδ4⁺ cells have the potential to produce Th1 and/or Th17 cytokines, and differences in the disease models may determine which type of cytokine is produced.

Two recent papers previously identified γδ T cells as a potent source of IL-17^{28,29}. Stark et al. found that in B6 mice, both αβ and γδ T cells produced IL-17. More recently, Lockhart et al., studying Mycobacterium tuberculosis infection of the mouse lung, found that γδ T cells were in fact the dominant source of IL-17, rather than CD4⁺ αβ T cells²⁹. The present inventors' results showed that in CIA, Vγ4/Vδ4⁺ cells predominated and the vast majority of these cells in both the draining lymph node and the joints of mice could be stimulated to produce IL-17. Moreover, there were as many IL-17⁺ Vδ4⁺ cells in the lymph node as CD4⁺ αβ TCR⁺IL-17⁺ cells. Based on studies in RA and EAE, IL-17 is now considered a major player in chronic autoimmune diseases. Studies in CIA have shown that disease is markedly suppressed in IL-17 “knocked-out” mice³⁰, and neutralization of IL-17 after the onset of CIA reduces joint inflammation, cartilage destruction and bone erosion³¹. Depleting Vγ4⁺ cells in our model and thus, removing a large source of IL-17, may explain why these mice had less severe arthritis and a lower incidence of disease.

Accordingly, one embodiment of the invention relates to a method to treat, such as by reducing the severity or incidence of, a condition or disease associated with the production of interleukin-17 (IL-17) by deleting, inactivating or inhibiting γδ T cells in an individual who has or is at risk of developing the disease. Various diseases and conditions are encompassed by the invention (see, e.g., Steinman, L., Nature Medicine 13:139, 2007, for a review of IL-17 and reference to diseases and conditions associated with this biological response modifier). The present inventors have discovered that γδ T cells, and in particular, subsets of γδ T cells, can contribute significantly to the production of IL-17 in certain tissues, conditions and diseases and can have a negative impact in some cases on the pathology of the disease by this mechanism. Such correlations have not previously been made, to the present inventors' knowledge in particular conditions such as autoimmune diseases, cancer, and various inflammatory conditions related to the mucosal organs and tissues. Examples of such conditions and diseases encompassed by the invention include, but are not limited to, cancer, rheumatoid arthritis, multiple sclerosis, systemic lupus erythematosus, asthma, yeast infections (e.g., Klebsiella or Candida infections), induction of granulopoiesis, allograft rejection, neutrophil migration to the lung, chronic enterocolitis, Bacteriodes fragilis, E. Coli infection, experimental myocarditis, and other microbial infections. In one embodiment, the method of the invention is useful for treating a cancer, including, but not limited to, melanomas, squamous cell carcinoma, breast cancers, head and neck carcinomas, thyroid carcinomas, soft tissue sarcomas, bone sarcomas, testicular cancers, prostatic cancers, pancreatic cancers, ovarian cancers, uterine cancers, cervical cancers, bladder cancers, skin cancers, brain cancers, angiosarcomas, hemangiosarcomas, mast cell tumors, primary hepatic cancers, lung cancers (including non-small cell lung carcinomas), pancreatic cancers, gastrointestinal cancers (including colorectal cancers), renal cell carcinomas, hematopoietic neoplasias and metastatic cancers thereof. In one aspect, the cancer is a cancer of a muscosal organ or tissue (e.g., uterine cancer, colorectal cancer). In one embodiment, the method of the invention is useful for treating an autoimmune disease, including, but not limited to, rheumatoid arthritis, multiple sclerosis, and systemic lupus erythematosus. In one embodiment, the method of the invention is useful for treating any inflammatory condition or disease of a muscosal tissue or organ, including any organ or tissue in the gastrointestinal tract or the reproductive tract. In one embodiment, the method of the invention is not used to treat a bacterial infection or a mycobacterial infection.

The methods of the invention include the step of deleting, inactivating or inhibiting γδ T cells in an individual who has or is at risk of developing the condition or disease. The present inventors have identified characteristics of γδ T cells, and particularly, a γδ T cell subset, that is believed to play a significant role in the pathogenesis, including the severity and incidence, of such conditions and diseases. Specifically, the subset of γδ T cells can be defined by one or more characteristics that can be targeted to selectively treat a condition or disease associated with IL-17. In one aspect, the subset of γδ T cells to be deleted, inactivated or inhibited produces IL-17. In one aspect, the subset of γδ T cells to be deleted, inactivated or inhibited both produce IL-17 and express activation markers. For example, the inventors have shown that IL-17 producing γδ T cells that are associated with the severity and incidence of CIA in the model of autoimmune arthritis described herein upregulate the T cell activation marker, CD44. In addition, the inventors have shown that these T cells also downregulate expression of CD62L and CD45RB, which is a phenotype that is also associated with activated T cells. These characteristics can be combined to identify the subset of γδ T cells to be deleted, inactivated or inhibited.

In additional embodiments of the invention, the subset of γδ T cells to be deleted, inactivated or inhibited can be identified based on the structure of its T cell receptor (TCR). The present inventors have identified this subset as having similar TCRs, both in terms of the Vγ and Vδ usage, but also in terms of the CDR3 regions of the receptors (i.e., these regions have highly conserved motifs), all indicating that the subset of γδ T cells to be targeted by the invention may represent an antigen-selected subset of cells. Accordingly, in another aspect of the invention, the subset of γδ T cells to be deleted, inactivated or inhibited produces IL-17 and has a T cell receptor comprised of the same Vγ and Vδ combination. In yet another aspect, the subset of γδ T cells to be deleted, inactivated or inhibited produces IL-17 and has a T cell receptor with a highly conserved amino acid motif in the CDR3 region of the TCR-δ chain. In yet another aspect, the subset of γδ T cells to be deleted, inactivated or inhibited produces IL-17 and has a T cell receptor with a highly conserved amino acid motif in the CDR3 region of the TCR-γ chain. In another aspect, the subset of γδ T cells to be deleted, inactivated or inhibited has a T cell receptor comprising Vγ4 or the human equivalent thereof. In yet another aspect, the subset of γδ T cells to be deleted, inactivated or inhibited has a T cell receptor comprising Vγ4 or the human equivalent thereof, and comprising Vδ4 or the human equivalent thereof. In targeting the γδ T cells to be deleted, inactivated or inhibited, cells having any combination of any of the characteristics described herein can be targeted.

A “γδ T cell” is a distinct lineage of T lymphocytes found in mammalian species and birds that expresses a particular antigen receptor (i.e., T cell receptor or TCR) that includes a γ chain and a δ chain. γδ T cell receptors are composed of a heterodimer of a γ chain and a δ chain. Multiple different functional murine γ chains, murine δ chains, human γ chains, and human δ chains are known, and the sequences of the chains are publicly available (see, e.g., Arden, et al. Immunogenetics 1995; Allison and Garboczi, Molecular Immunol. 38: 1051-1061, 2002; Konihshofer and Chien, Current Opinion of Immunol. 18: 527-533, 2006). The γ and δ chains are distinguished from the α and β chains that make up the TCR of the perhaps more commonly referenced T cells known as “αβ T cells”. The γδ heterodimer of the γδ T cells is expressed on the surface of the T cell and, like the αβ heterodimer of αβ T cells, is associated with the CD3 complex on the cell surface. The γ and δ chains of the γδ T cell receptor should not be confused with the γ and δ chains of the CD3 complex. According to the present invention, the terms “T lymphocyte” and “T cell” can be used interchangeably herein.

Interleukin-17 (IL-17) is T cell-derived, proinflammatory cytokine that is suspected to be involved in the development of various inflammatory diseases, as discussed above. Numerous immune regulatory functions have been reported for the IL-17 family of cytokines, presumably due to their induction of many immune signaling molecules. IL-17 is primarily noted for its involvement in inducing and mediating proinflammatory responses. IL-17 is commonly associated with allergic responses, has been implicated in the pathogenesis of various inflammatory and autoimmune diseases, and IL-17 induces the production of many other cytokines and chemokines (e.g. IL-6, IL-8, G-CSF, GM-CSF, IL-1β, TGF-β, TNF-α, GRO-α and MCP-1) and prostaglandins in a variety of cells.

According to the invention, a variety of techniques can be used to delete, inactivate or inhibit the targeted γδ T cells. For example, in one aspect, a common ex vivo technique for removing components from the blood, and then returning the blood to the individual, known as leukophoresis, is used. The use of leukophoresis to selectively remove components from the blood, for example, by using binding agents such as antibodies or soluble receptors, has been described (e.g., see U.S. Pat. No. 6,379,708). In the present invention, blood can be removed from a patient to be treated, the blood treated to selectively remove the targeted γδ T cells, and then the blood is returned to the patient, free of γδ T cells that are contributing to disease processes. For example, γδ T cells, or selected subsets thereof, could be removed from the blood through the use of immobilized antibodies or other binding moieties that selectively bind to the targeted γδ T cell (e.g., antibodies that selectively bind to a particular Vδ chain, a particular Vγ chain), and thereby remove it from the blood. However, since the γδ T cells causing damage to the patient may be localized to a tissue or organ, other methods for the deletion, inactivation or inhibition of such cells may also be utilized and in some instances, may be preferable.

Another method of deleting, inactivating or inhibiting the targeted γδ T cells include the administration of an agent that contacts the targeted γδ T cell, or a ligand with which the targeted γδ T cell interacts, resulting in the deletion of the γδ T cell, inactivation of the γδ T cell, or the inhibition of the γδ T cell. Inhibition can be achieved by directly inhibiting the activity of the targeted γδ T cell, or by inhibiting a biological activity of the γδ T cell by blocking or inhibiting the interaction of the T cell with, for example, its natural ligand, or by inhibiting the activity of a mediator released by the T cell (e.g., IL-17).

According to the present invention, the phrase “selectively binds to” refers to the ability of an antibody, antigen-binding fragment or binding partner of the present invention to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen-binding fragment thereof in the absence of the antigen is considered to be background. Binding can be measured using a variety of methods standard in the art including enzyme immunoassays (e.g., ELISA), immunoblot assays, etc.). To “selectively target” a given moiety, such as a receptor, refers to the specific targeting of an agent, treatment or action to a specific moiety, such as by selective binding of an agent to the moiety, or by applying a protocol that will selectively act on the moiety and not substantially on another, different moiety.

Accordingly, in one aspect of the invention, the targeted γδ T cells are deleted, inactivated or inhibited by administration of an agent that selectively targets γδ T cells having a specified Vγ and Vδ combination. In other embodiments, the agent can selectively target γδ T cells expressing TCR-Vδ4, or the human equivalent thereof, or selectively targets γδ T cells expressing TCR-Vγ4/Vδ4, or the human equivalent thereof. One type of agent that is particularly useful in these aspects of the invention includes, but is not limited to, antibodies or antigen-binding fragments thereof, or any binding protein or molecule (e.g., an aptamers) that selectively binds to the targeted TCR.

Antibodies are characterized in that they comprise immunoglobulin domains and as such, they are members of the immunoglobulin superfamily of proteins. An antibody of the invention includes polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)² fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Genetically engineered antibodies of the invention include those produced by standard recombinant DNA techniques involving the manipulation and re-expression of DNA encoding antibody variable and/or constant regions. Particular examples include, chimeric antibodies, where the VH and/or VL domains of the antibody come from a different source to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

The invention also extends to non-antibody polypeptides, sometimes referred to as binding partners or antigen-binding polypeptides, that have been designed to bind specifically to a given protein. Examples of the design of such polypeptides, which possess a prescribed ligand specificity are given in Beste et al. (Proc. Natl. Acad. Sci. 96:1898-1903, 1999), incorporated herein by reference in its entirety.

Another useful agent in the methods of the invention is a soluble γδ T cell receptor or any competitive inhibitor that competitively inhibits the binding of the natural γδ T cell receptor to its ligand. According to the present invention, a competitive inhibitor of γδ TCR is an inhibitor that binds to the same or similar epitope of a ligand (antigen) of the γδ TCR as the γδ TCR, such that binding of the γδ TCR to its ligand is inhibited. A competitive inhibitor may bind to the target ligand with a greater affinity for the target ligand than the γδ TCR.

Soluble γδ TCRs are described in detail in PCT Publication No. WO 03/060097, which is incorporated herein by reference in its entirety. According to the present invention, a “soluble” T cell receptor is a T cell receptor consisting of the chains of a full-length (e.g., membrane bound) receptor, except that, minimally, the transmembrane region of the receptor chains are deleted or mutated so that the receptor, when expressed by a cell, will not associate with the membrane. Most typically, a soluble receptor will consist of only the extracellular domains of the chains of the wild-type receptor (i.e., lacks the transmembrane and cytoplasmic domains). Various specific combinations of γ and δ chains are preferred for use in the soluble γδ T cell receptors, and particularly those corresponding to γδ T cell subsets that are known to exist in vivo, and more particularly those corresponding to γδ T cell subsets that are targeted for inhibition according to the invention. However, it is to be understood that soluble γδ T cell receptors having virtually any combination of γ and δ chains are also contemplated for use in the present invention. Preferably, soluble γδ T cell receptors comprise γ and δ chains derived from the same animal species (e.g., murine, human).

A soluble γδ T cell receptor useful in the invention typically is a heterodimer comprising a γ chain and a δ chain, but multimers (e.g., tetramers) comprising two different γδ heterodimers or two of the same γδ heterodimers are also contemplated for use in the present invention. As set forth above, preferably, γ and δ chains from the same species of mammal (e.g., murine, human) are combined to form a γδ heterodimer.

Another useful agent is a soluble IL-17 receptor, which could be targeted (e.g., by producing a chimeric protein) to a tissue or to a γδ T cell, or simply administered to the site of γδ T cell activation. Since αβ T cells also produce IL-17, and since it may be desirable in some embodiments to preserve the activity of these cells while inhibiting γδ T cells, it is preferable to select a method that targets γδ T cells, and especially the subset of γδ T cells associated with the pathology of the disease to be treated. IL-17 receptors are known in the art.

The invention also includes small molecule compounds that can serve as inhibitors of the γδ T cell subset according to the invention (e.g., products of drug discovery). Such an agent can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the same building blocks) or by rational drug design. See for example, Maulik et al., 1997, Molecular Biotechnology: Therapeutic Applications and Strategies, Wiley-Liss, Inc., which is incorporated herein by reference in its entirety. Candidate compounds initially identified by drug design methods can be screened for the ability to modulate γδ T cell activity as described herein.

Any of the agents for the deletion, inactivation, or inhibition of γδ T cells described herein can be administered to an individual in the form of a therapeutic composition. Therapeutic compositions can also contain one or more pharmaceutically acceptable excipients, and/or one or more additional agents useful for treating a particular disease or condition (e.g., one or more agents suitable for use in treating an autoimmune disease, such as rheumatoid arthritis). As used herein, a pharmaceutically acceptable excipient refers to any substance suitable for delivering a therapeutic composition useful in the method of the present invention to a suitable in vivo or ex vivo site. Preferred pharmaceutically acceptable excipients are capable of maintaining a composition in a form that, upon arrival of the composition at a target cell, tissue, or site in the body, the therapeutic agent(s) is capable of acting in the intended manner. Suitable excipients of the present invention include excipients or formularies that transport, but do not specifically target a composition to a site (also referred to herein as non-targeting carriers). Examples of pharmaceutically acceptable excipients include, but are not limited to water, saline, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol.

The present invention includes the delivery of an agent or composition to an individual. The administration process can be performed ex vivo or in vivo. Ex vivo administration refers to performing part of the regulatory step outside of the patient, such as by removing cells from a patient, treating them to remove or inactivate the target γδ T cells, and then returning the remaining cells to the patient.

Administration of an agent or composition can be systemic, mucosal and/or proximal to the location of the target site (e.g., to the joints of an individual). The preferred routes of administration will be apparent to those of skill in the art, depending on the type of condition to be prevented or treated, the agent used, and/or the target cell population or tissue. Preferred methods of administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, aural, intranasal, oral, pulmonary administration, impregnation of a catheter, and direct injection into a tissue. Particularly preferred routes of administration include: intravenous, intraperitoneal, subcutaneous, intradermal, intranodal, intramuscular, transdermal, inhaled, intranasal, oral, intraocular, intraarticular, intracranial, and intraspinal. Parenteral delivery can include intradermal, intramuscular, intraperitoneal, intrapleural, intrapulmonary, intravenous, subcutaneous, atrial catheter and venal catheter routes. Aural delivery can include ear drops, intranasal delivery can include nose drops or intranasal injection, and intraocular delivery can include eye drops. Aerosol (inhalation) delivery can also be performed using methods standard in the art (see, for example, Stribling et al., Proc. Natl. Acad. Sci. USA 189:11277-11281, 1992, which is incorporated herein by reference in its entirety). For example, in one embodiment, a composition can be formulated into a composition suitable for nebulized delivery using a suitable inhalation device or nebulizer. Oral delivery can include solids and liquids that can be taken through the mouth.

According to the present invention, an effective administration protocol (i.e., administering an agent or therapeutic composition in an effective manner) comprises suitable dose parameters and modes of administration that result in the deletion, inactivation, or inhibition of the γδ T cells as described herein, preferably so that the individual receiving the treatment is provided with some benefit as a result of the administration. Preferably, the administration results in the alleviation or detectable improvement in at least one symptom or indicator of the disease or condition in the individual, such as reduced incidence of disease, or decreased severity of at least one symptom of the disease. Effective dose parameters can be determined using methods standard in the art for a particular disease. Such methods include, for example, determination of survival rates, side effects (i.e., toxicity) and progression or regression of disease.

Methods and uses directed to therapeutic agents and compositions of the invention are primarily intended for use in the prevention and/or treatment of a disease or condition. A therapeutic composition or agent of the present invention, when administered to an individual, can: prevent a disease from occurring; cure the disease; delay the onset of the disease; reduce the incidence of the disease; and/or alleviate (reduce, delay, diminish) disease symptoms, signs or causes (e.g., reduce one or more symptoms of the disease; reduce the occurrence of the disease; increase survival of the individual that has or develops the disease; and/or reduce the severity of the disease).

Diseases to be treated using the methods of the invention include any disease in which IL-17 mediates or contributes to the pathogenesis of the disease, and even more particularly, any disease in which IL-17 producing γδ T cells, and more particularly, a subset of such cells, contribute to the pathogenesis of the disease (e.g., by being a significant source of IL-17). Such diseases include, but are not limited to, cancer, autoimmune diseases, and inflammatory conditions of the mucosal tissues and organs. In one aspect, such diseases include, but are not limited to: rheumatoid arthritis, multiple sclerosis, systemic lupus erythematosus, asthma, yeast infections (e.g., Klebsiella or Candida infections), induction of granulopoiesis, allograft rejection, neutrophil migration to the lung, chronic enterocolitis, Bacteriodes fragilis, E. Coli infection, experimental myocarditis, and other microbial infections. In one embodiment, the method of the invention is useful for treating a cancer, including, but not limited to, melanomas, squamous cell carcinoma, breast cancers, head and neck carcinomas, thyroid carcinomas, soft tissue sarcomas, bone sarcomas, testicular cancers, prostatic cancers, pancreatic cancers, ovarian cancers, uterine cancers, cervical cancers, bladder cancers, skin cancers, brain cancers, angiosarcomas, hemangiosarcomas, mast cell tumors, primary hepatic cancers, lung cancers (including non-small cell lung carcinomas), pancreatic cancers, gastrointestinal cancers (including colorectal cancers), renal cell carcinomas, hematopoietic neoplasias and metastatic cancers thereof. In one aspect, the cancer is a cancer of a muscosal organ or tissue (e.g., uterine cancer, colorectal cancer). In one embodiment, the method of the invention is useful for treating an autoimmune disease, including, but not limited to, rheumatoid arthritis, multiple sclerosis, and systemic lupus erythematosus. In one embodiment, the method of the invention is useful for treating any inflammatory condition or disease of a muscosal tissue or organ, including any organ or tissue in the gastrointestinal tract or the reproductive tract. In one embodiment, the method of the invention is not used to treat a bacterial infection or a mycobacterial infection.

Another embodiment of the present invention relates to a method to identify an agent useful for the treatment of any disease or condition described herein that is associated with the production of interleukin-17 (IL-17). In one aspect, the method includes: (a) contacting γδ T cells that produce IL-17 with a putative agent, wherein the γδ T cells were obtained or derived from a patient with such a disease associated with the production of IL-17; and (b) selecting a putative agent that deletes, inactivates or inhibits the γδ T cells of (a) as an agent for the treatment of the disease. In another aspect, the method includes: (a) contacting γδ T cells that produce IL-17 and that express TCR-Vγ4, or the human equivalent thereof, with a putative agent; and selecting a putative agent that deletes, inactivates or inhibits the γδ T cells of (a) as an agent for the treatment of the disease. In other aspects of this embodiment, one may also use non-cell based assays, including the use of cell lysates, isolated receptors, and nucleic acids, in order to screen for compounds with putative activity, which can be followed with functional, e.g., cell based or non-human animal assays, to confirm the properties of the compound.

In this embodiment of the invention, the γδ T cells can be obtained or derived from any suitable source. Cells can include primary cell isolates, cell lines, and immortalized cell lines, as well as recombinantly produced cells. In the case of cells derived from a patient, the cells can be obtained or derived from a patient with any disease to be treated using the methods of the invention. Such diseases are described above.

Compounds or putative agents to be screened in the methods of the invention include known organic compounds such as products of peptide libraries, nucleic acid molecules (e.g., RNAi, ribozymes, aptamers, anti-sense), antibodies, and products of chemical combinatorial libraries. Compounds may also be identified using rational drug design relying on the structure of the product of a gene, alone or in complex with another component. Such methods are known to those of skill in the art and involve the use of three-dimensional imaging software programs. Various methods of drug design, useful to design or select mimetics or other therapeutic compounds useful in the present invention are disclosed in Maulik et al., 1997, supra, which is incorporated herein by reference in its entirety.

As used herein, the term “test compound or agent”, “putative inhibitory compound or agent” or “putative regulatory compound or agent” refers to compounds having an unknown or previously unappreciated regulatory activity in a particular process. As such, the term “identify” with regard to methods to identify compounds is intended to include all compounds, the usefulness of which as a regulatory compound for the purposes of inhibiting γδ T cells is determined by a method of the present invention.

The conditions under which a cell, cell lysate, nucleic acid molecule or protein of the present invention is exposed to or contacted with a putative regulatory compound, such as by mixing, are any suitable culture or assay conditions. In the case of a cell-based assay, the conditions include an effective medium in which the cell can be cultured or in which the cell lysate can be evaluated in the presence and absence of a putative regulatory compound. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Cells are contacted with a putative regulatory compound under conditions which take into account the number of cells per container contacted, the concentration of putative regulatory compound(s) administered to a cell, the incubation time of the putative regulatory compound with the cell, and the concentration of compound administered to a cell. Determination of effective protocols can be accomplished by those skilled in the art based on variables such as the size of the container, the volume of liquid in the container, conditions known to be suitable for the culture of the particular cell type used in the assay, and the chemical composition of the putative regulatory compound (i.e., size, charge etc.) being tested. A preferred amount of putative regulatory compound(s) can comprise between about 1 nM to about 10 mM of putative regulatory compound(s) per well of a 96-well plate.

One aspect of this embodiment of the invention comprises selecting the putative agent. In one aspect, the step of selecting comprises selecting a putative agent that inhibits the production of IL-17 by the γδ T cells. In one aspect, the γδ T cells express TCR-Vγ4/Vδ4, or the human equivalent thereof. Any suitable method of selecting for the activity of an agent on γδ T cells or a portion thereof (the γδ T cell receptor) is suitable for use in the invention. Accordingly, one may select an agent based on the effect of the agent on a variety of parameters including, but not limited to, γδ T cell proliferation, production of cytokines and factors by the γδ T cells, upregulation or downregulation of expression of cellular markers of activation or apoptosis, binding to a receptor on the cell, and/or regulation of gene expression by the cell.

T cell proliferation assays, including those using γδ T cells, are well known in the art, and are described, for example, in several publications by certain of the present inventors (e.g., Born et al., 1990, Science 249:67; O'Brien et al., 1992, Proc. Natl. Acad. Sci. USA 89:4348; Lahn et al., 1998, J. Immunol. 160:5221; Cady et al., 2000, J. Immunol. 165:1790; all incorporated herein by reference in their entireties). Other methods for evaluating γδ T cells include detecting or measuring the expression level and/or the distribution of γ-chain usage and/or δ chain usage in the receptors of a population of γδ T cells, and determining whether there is a change in the expression level and/or distribution of one or more γδ T cell receptor types in the population. Such assays, including both molecular and flow cytometric methods, and the reagents (e.g., antibodies, hybridization probes and PCR primers specific for various γδ TCR chains) for performing such assays, are known in the art (e.g., O'Brien et al., 1992, supra; Lahn et al., 1998, supra; Cady et al., 2000, supra).

Activation, or responsiveness, of a γδ T cell refers to the ability of a γδ T cell to be activated by (e.g., respond to) antigenic and/or mitogenic stimuli which results in induction of γδ T cell activation signal transduction pathways and activation events. The biological activity of a γδ T cell refers to any function(s) exhibited or performed by a naturally occurring γδ T cell as measured or observed in vivo (i.e., in the natural physiological environment of the cell) or in vitro (i.e., under laboratory conditions). As used herein, antigenic stimulation is stimulation of a γδ T cell by binding of the γδ T cell receptor to an antigen that is specifically recognized by the γδ T cell in the context of appropriate costimulatory signals necessary to achieve γδ T cell activation. Mitogenic stimulation is defined herein as any non-antigen stimulation of T cell activation, including by mitogens (lipopolysaccharides (LPS), phorbol esters, ionomycin) and antibodies (anti-TCR, anti-CD3, including divalent and tetravalent antibodies). Both antigenic stimulation and the forms of mitogenic stimulation which act at the level of the T cell receptor (i.e., anti-TcR/CD3) result in T cell receptor-mediated activation, whereas LPS/phorbol ester/ionomycin mitogenic stimulation bypasses the T cell receptor and therefore, do not induce T cell receptor-mediated activation, but nonetheless, can induce at least some of the downstream events of T cell activation.

Therefore, events associated with T cell activation or biological activity include, but are not limited to, T cell proliferation, cytokine production (e.g., interleukin-2 (IL-2), IL-4, IL-5, IL-10, IL-17, interferon-γ (IFN-γ), tumor necrosis factor-α (TNF-α)), upregulation of cytokine receptors (e.g., IL-2 receptor, TNF-α receptor), calcium mobilization, upregulation of cell surface molecules associated with T cell activation (e.g., CD44, CD69), upregulation of expression and activity of signal transduction proteins associated with T cell activation, chemokine production, altered T cell migration, accumulation of T cells at specific tissue sites and/or cytoskeletal reorganization. The ability of a T lymphocyte to respond, or become activated, by an antigenic or mitogenic stimulus can be measured by any suitable method of measuring T cell activation. Such methods are well known to those of skill in the art. For example, after a T cell has been stimulated with an antigenic or mitogenic stimulus, characteristics of T cell activation can be determined by a method including, but not limited to: measuring cytokine production by the T cell (e.g., by immunoassay or biological assay); measuring intracellular and/or extracellular calcium mobilization (e.g., by calcium mobilization assays); measuring T cell proliferation (e.g., by proliferation assays such as radioisotope incorporation); measuring upregulation of cytokine receptors on the T cell surface, including IL-2R (e.g., by flow cytometry, immunofluorescence assays, immunoblots, RNA assays); measuring upregulation of other receptors associated with T cell activation on the T cell surface (e.g., by flow cytometry, immunofluorescence assays, immunoblots, RNA assays); measuring reorganization of the cytoskeleton (e.g., by immunofluorescence assays, immunoprecipitation, immunoblots); measuring upregulation of expression and activity of signal transduction proteins associated with T cell activation (e.g., by kinase assays, phosphorylation assays, immunoblots, RNA assays); and, measuring specific effector functions of the T cell (e.g., by proliferation assays). Methods for performing each of these measurements are well known to those of ordinary skill in the art, many are described in detail or by reference to publications herein, and all such methods are encompassed by the present invention.

In the methods of the invention, a selected agent is selected on the basis of a particular action or activity, as compared to a suitable control.

Candidate compounds identified or designed by the above-described methods can be synthesized using techniques known in the art, and depending on the type of compound.

Any compound identified by these methods can be used to treat or prevent any disease or condition as described herein, or in other methods or uses described herein.

An “individual” may be a vertebrate, preferably a mammal, more preferably a human. Mammals include, but are not limited to, farm animals, sport animals, pets, primates, mice and rats. The term “individual” can be used interchangeably with the term “animal”, “subject” or “patient”.

The following experimental results are provided for purposes of illustration and are not intended to limit the scope of the invention.

### EXAMPLES

### Materials and Methods

Animals 8-10 week old DBA/1 lac J male mice (Jackson Laboratories, Bar Harbor, Me.) were used for this study.

Immunizations Bovine type II collagen (CII) (Elastin Products, Owensville, Mo.) was diluted in 0.01M acetic acid to a final concentration of 4 mg/ml and stored at −70° C. Before injection, an equal volume of CII was emulsified with Freund's incomplete adjuvant (Difco, Detroit, Mich.), to which 4 mg/ml inactivated Mycobacterium tuberculosis (H37Ra; Difco) had been added to generate Complete Freund's Adjuvant (CFA). The emulsion was kept on ice during both preparation and use. The mice were injected intradermally at the base of the tail with 100 μl of the emulsion, containing 200 μg of CII and 200 μg of M. tuberculosis, on day 0 and day 21. Mice were scored for severity of disease every other day starting on day 21 until they were sacrificed on day 41. The following scale was used: 0, no redness or swelling; 1, 1 digit swollen; 2, 2 digits swollen; 3, 3 digits swollen; and 4, entire paw swollen with ankylosis. The scores for each of 4 paws were added together to give a final score, such that the maximal severity score was 16.

Analysis of γδ T cells Throughout these examples, the inventors have used the simple numbering system of Heilig and Tonegawa for the murine γ and δ genes³². Official nomenclature equivalents are shown in parentheses²²: Vγ1 (GV5S1), Vγ4 (GV3S1), Vδ4 (DV104S1), Vδ5 (DV105S1), and Vδ6.3 (ADV7S1). On various days after the first or second collagen/CFA immunization, mice were sacrificed, and the draining (inguinal, popliteal, and brachial) lymph nodes removed for flow cytometric analysis. A cell suspension from the lymph nodes was made using mesh screens, and T cells were enriched by passage over nylon wool³³. Nylon wool non-adherent cells were stained for γδ T cells subsets using a FITC-labeled pan Cδ antibody (GL3,³⁴), followed by biotinylated anti-Vγ1 (2.11,¹³) or anti-Vγ4 (UC3-10A6,³⁵) antibodies plus strepavidin-APC, and PE-conjugated anti-CD62L, anti-CD45RB, or anti-CD44 antibodies (BD Biosciences, San Jose, Calif.). All samples were analyzed on a FACScalibur or FACScan flow cytometer (Becton Dickinson, Franklin Lakes, N.J.), and the data processed using FlowJo 6.4.1 software (Tree Star, Inc. Stanford, Calif.).

Treatment with depleting antibodies Mice were injected with 200 μg of either an anti-Vγ4 antibody (UC3), an anti-Vγ1 antibody (clone 2.11), or with hamster IgG, as a control, on day 17, four days before the booster immunization with CII/CFA. On day 21, blood lymphocytes were tested to check for the depletion of the appropriate γδ T cell subset by flow cytometry. Briefly, heparinized blood samples were incubated in Gey's solution for 10 minutes to lyse the red blood cells. The remaining cells were passed over nylon-wool columns in order to enrich for T cells. Nylon wool non-adherent cells were then stained with anti-CD3 (KT3,³⁶), an anti-Cδ antibody (GL3) and either anti-Vγ1 (2.11) or anti-Vγ4 (UC3) antibodies to verify the depletion of the appropriate subset. All samples were analyzed on a FACScan or FACScalibur flow cytometer and the data processed using FlowJo 6.4.1 software.

Measurement of anti-collagen antibodies Serum from each mouse was obtained by aspiration of retroorbital blood on days 0 and 21, at the time of the first and just before the second CII injection. On day 41, mice were tail bled before being sacrificed. Each serum sample was analyzed for the level of total IgG, IgG1, and IgG2a antibodies to type II collagen using modifications of published ELISA methods. Briefly, Immunlon II ELISA plates were coated with 5 μg/ml of CII (Chondrex Seattle, Wash.) overnight at 4° C. Plates were washed three times with PBS containing 0.05% Tween and 1% bovine serum albumin (BSA), then blocked with PBS and 1% BSA at 4° C. for 4 hours. The blocking solution was removed and 50 μl of a 1:9000 dilution of each serum sample was added in duplicate and the plates were incubated overnight at 4° C. The plates were washed with PBS containing 0.05% Tween, and 50 μl of horseradish peroxidase-conjugated goat-anti mouse IgG (diluted 1:3000 in PBS), IgG1 (diluted 1:2000), or IgG2a (diluted 1:2000) antibody (Invitrogen, Carlsbad, Calif.) was added to each well and incubated for 4 hours at 4° C. The plates were washed again with PBS containing 0.05% Tween before 50 μl of TMB substrate was added. The plates were developed for 5 minutes before the reaction was stopped with the addition of 25 μl of 2N H₂SO₄. Absorbance was measured at 450 nm on a VERSAmax microplate reader and the data analyzed using Softmax Pro 4.7.1 software (Molecular Devices, Sunnyvale, Calif.). A standard pool of anti-collagen antibodies was obtained by combining sera from several mice with severe disease. The levels of IgG, IgG1, and IgG2a anti-collagen antibodies in this pool of sera were set as equivalent to 1000 units/ml.

Histology On day 41, forepaws and hind paws (including the paw and ankle) were surgically removed and fixed immediately in 10% buffered formalin. Tissue was prepared and histological analyses were performed as previously described³⁷. The treatment and clinical disease activity score of each sample was not disclosed to the trained observer who scored the slides. Sections were scored for mean inflammation, pannus formation, cartilage damage, and bone damage, and the overall score was based on a set of 3-4 joints per animal. All were scored on a 0-5 scale, as previously described³⁷.

Isolation of cells from the joint A modified version of a lung digestion protocol was used to obtain a cell suspension from the joints of mice³⁸. Briefly, the skin was first removed from the mouse paws and then the paws were dissected into small pieces. The pieces were placed in an enzymatic digestion mixture containing 0.125% dispase II (Roche, Indianapolis, Ind.), 0.2% collagenase II (Sigma-Aldrich, St. Louis, Mo.), and 0.2% collagenase IV (Sigma-Aldrich) and shaken for 75 min at 37° C. After digestion, the supernatant was removed and the joint pieces were pushed through a cellector tissue sieve (Bellco Glass, Inc. Vineland, N.J.) to disperse the cells. The cell suspension was then treated with Gey's solution to remove red blood cells and passed over nylon wool columns to enrich for T cells.

Intracellular Cytokine staining Nylon wool non-adherent cells were cultured at 1×10⁶ cells/ml in culture medium³⁹ containing 10 μg/ml Brefeldin A (Sigma-Aldrich), 50 ng/ml PMA (Sigma-Aldrich), and 1 μg/ml ionomycin (Sigma-Aldrich) at 37° C. for 4 hours. After activation, cells were washed once in staining buffer and then stained with anti-CD3-APC-AF750 (EBioscience, San Diego, Calif.), FITC-labeled anti-TCRδ (GL3), biotinylated or FITC-labeled anti-Vγ1 (2.11) or anti-Vγ4 (UC3-10A6), and biotinylated anti-Vδ4 (GL2,³⁴), anti-Vγ5 (F45.152,⁴⁰), or anti-Vδ6.3 (17C,⁴¹) antibodies and detected with strepavidin-APC (BD Biosciences). The cells were then fixed in 1% paraformaldehyde for at least 20 minutes at 4° C. Fixed cells were permeabilized for 10 minutes at 4° C. in 5% saponin/PBS buffer. Cells were then spun down and stained with PE-conjugated anti-cytokine antibodies (IL-2, IL-17, IFNγ, and TNFα; BD Biosciences) or an isotype control for 30 minutes at 4° C. Cells were washed once in saponin buffer and once in staining buffer before fixation in 1% paraformaldehyde. Samples were analyzed on a FACScan (Becton Dickinson) and the data processed using FlowJo v6.4.1 software.

Statistical analyses All statistical analyses were performed using GraphPad Prism version 4 (GraphPad Software, San Diego, Calif.). Statistical significance for the clinical disease activity was determined using the Mann-Whitney test. The histological data were analyzed by comparing group means using the Student's t-test with significance set at 5%. For the anti-collagen antibodies, statistical significance was determined using an unpaired 2-tailed Student's t-test to compare the two treatment groups.

Sequencing of TCRs Total cell RNA was isolated from nylon wool non-adherent cells obtained from the lymph nodes of mice using the PicoPure RNA Isolation Kit (Arcturus Bioscience, Mountain View, Calif.). Reverse transcription-PCR was performed using primers specific for Vδ4/Cγ1, 2 and Vδ4/Cδ as previously described³⁹. PCR-amplified transcripts were then cloned into a TA vector (Invitrogen, Carlsbad, Calif.) and individual clones sequenced to determine the frequency of specific TCR sequences.

### Example 1

The following example demonstrates that γδ T cell subsets respond differentially in CIA.

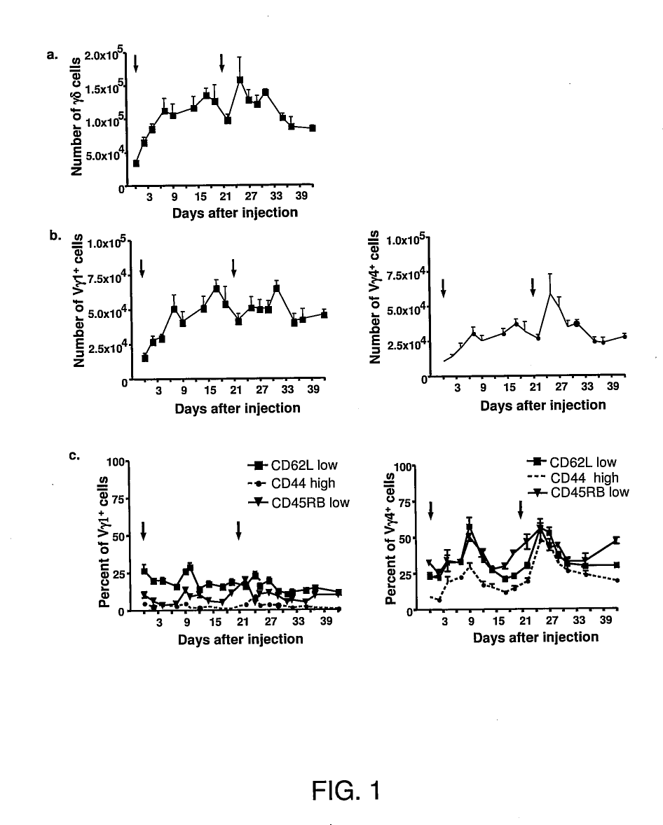

To further define the role of γδ T cells in CIA, the inventors analyzed the two main lymphoid γδ T cell subsets in mice on various days after collagen/CFA injection. Nine days after the first injection, total γδ T cells were increased approximately three-fold when compared to untreated mice (day 0) (FIG. 1a). Within 3-4 days following the second immunization, total γδ T cells increased again (FIG. 1a). The responses of both the Vγ1⁺ and Vγ4⁺ γδ T cells mirrored that of total γδ T cells, and both increased in numbers to approximately the same degree after the first collagen/CFA injection. However, Vγ4⁺ cells increased rapidly after the second injection, while Vγ1⁺ cells increased more slowly and less vigorously (FIG. 1b).

The loss of CD62L and CD45RB expression along with the gain of CD44 have been shown to correlate with αβ T cell activation/memory¹⁶. Therefore, the inventors also stained the γδ T cell subsets for these markers at various time points after CII immunization. As shown in FIG. 1c, the percentage of Vγ4⁺ cells that expressed high levels of CD44 increased (more than 10 fold) within the first 9 days of the disease course. A reciprocal loss of CD62L and CD45RB expression was also seen. These “activated” cells were transient and returned to near-baseline levels during the first three weeks of the disease process. Following the second immunization, the percent of “activated” Vγ4⁺ cells again increased. In contrast, Vγ1⁺ cells exhibited little change in expression of CD44, CD45RB and CD62L, even though Vγ1⁺ cell numbers increased during CIA (FIG. 1c). Therefore, the Vγ4⁺ subset appeared to be specifically responsive to the immunizations, whereas the Vγ1⁺ subset did not.

### Example 2

The following example demonstrates that Vδ4⁺γδ T cells are pathogenic.

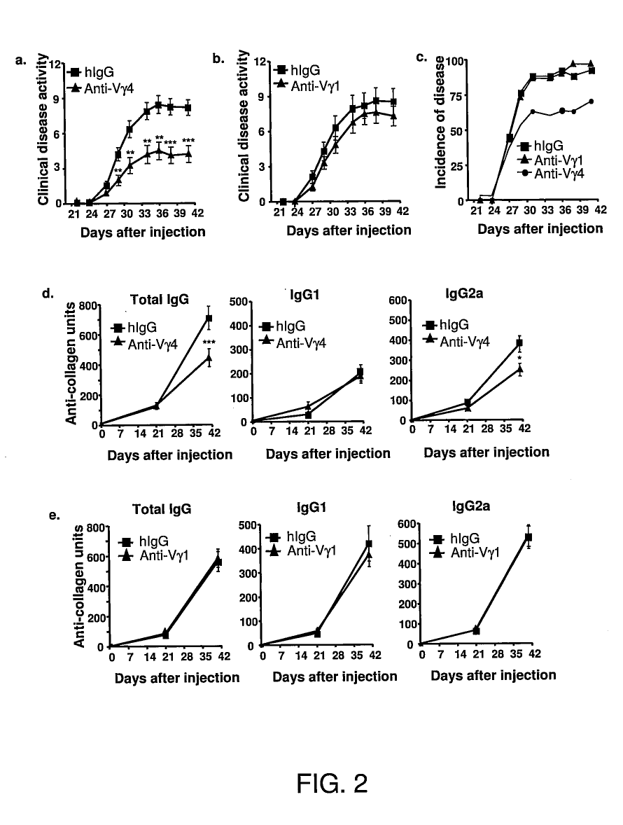

In order to determine the contribution of the Vγ1⁺ and Vγ4⁺ subsets to the development of CIA, mice were injected intravenously on day 17 with an anti-Vγ4 mAb or anti-Vγ1 mAb, to deplete the Vγ4⁺ or Vγ1⁺ subset, respectively, before the second injection of collagen/CFA. A control group of mice, injected with hamster IgG, was included in each experiment. Less than 1% of the relevant subset remained detectable in the blood after depletion (data not shown). As shown in FIG. 2a, Vγ4-depleted mice showed significantly less clinical disease as compared to control mice. In contrast, clinical disease scores were not significantly changed in Vγ1-depleted mice (FIG. 2b). The overall incidence of disease was also lower in the Vγ4-depleted mice but not in the Vγ1-depleted animals (FIG. 2c). On day 41, the mice were sacrificed and the joints from the Vγ4-depleted, Vγ1-depleted, and hamster IgG treated mice were examined for changes in inflammation, pannus, cartilage damage and bone damage. The Vγ4-depleted mice showed a 42% decrease in total score for all histological parameters examined when compared with those obtained from hamster IgG treated mice (Table 1). In agreement with the overall disease scores, Vγ1-depleted mice showed no statistical difference in any of their histological scores when compared to control mice (Table 1).

TABLE 1
Histopathology scores in mice with collagen-induced arthritis treated
with either an anti-Vγ4 antibody or an anti-Vγ1 antibody*

| | Hamster IgG | Anti-Vγ4 | |
|---|---|---|---|
| Parameter | (26 mice) | (30 mice) | P† |
| Inflammation | 2.59 ± 0.24 | 1.57 ± 0.27 | 0.007 |
| Pannus | 2.13 ± 0.25 | 1.20 ± 0.22 | 0.007 |
| Cartilage damage | 2.55 ± 0.24 | 1.47 ± 0.26 | 0.004 |
| Bone damage | 2.13 ± 0.25 | 1.20 ± 0.22 | 0.007 |
| Total score | 9.38 ± 0.97 | 5.43 ± 0.96 | 0.005 |
| | Hamster IgG | Anti-Vγ1 | |
| Parameter | (25 mice) | (30 mice) | P† |
| Inflammation | 2.43 ± 0.37 | 2.31 ± 0.26 | 0.783 |
| Pannus | 1.86 ± 0.29 | 1.65 ± 0.21 | 0.553 |
| Cartilage damage | 2.43 ± 0.38 | 2.22 ± 0.27 | 0.642 |
| Bone damage | 1.86 ± 0.29 | 1.65 ± 0.21 | 0.553 |
| Total score | 8.58 ± 1.33 | 7.83 ± 0.94 | 0.637 |

*The inflammation, pannus, cartilage damage, and bone damage scores were determined for each joint examined (3-4 per animal). Changes were scored on a 0-5 scale. A mean score for each animal was determined for each parameter, and these were averaged to determine group means. Values shown are the group means ± SEM from 2 separate experiments. Statistical analysis of histopathologic parameters were done by comparing group means using the Student's t test with significance set at 5%.
†anti-Vγ4 or anti-Vγ1 versus hamster IgG treatment.

Next, total IgG, IgG1 and IgG2a anti-collagen antibody levels were measured in the sera from the treated and control mice to determine if γδ T cell subsets contributed to anti-collagen antibody production. No anti-collagen antibodies were detectable on day 0. On day 21, four days after the anti-Vγ4 or anti-Vγ1 treatment was given, the levels of total IgG, IgG1, and IgG2a anti-collagen antibodies were still equivalent to those seen in hamster IgG-treated control animals. However, by day 41 there was a significant decrease in the total IgG and pathogenic IgG2a levels of anti-collagen antibodies in the Vγ4-depleted mice (FIG. 2d). In contrast, mice depleted of Vγ1⁺ cells showed no change in antibody levels (FIG. 2e). The level of IgG1 anti-collagen antibodies did not differ from control groups in either Vγ4-depleted or Vγ1-depleted animals.

### Example 3

The following example demonstrates that Vδ4⁺ γδ T cells produce IL-17 in the draining lymph nodes and joints.

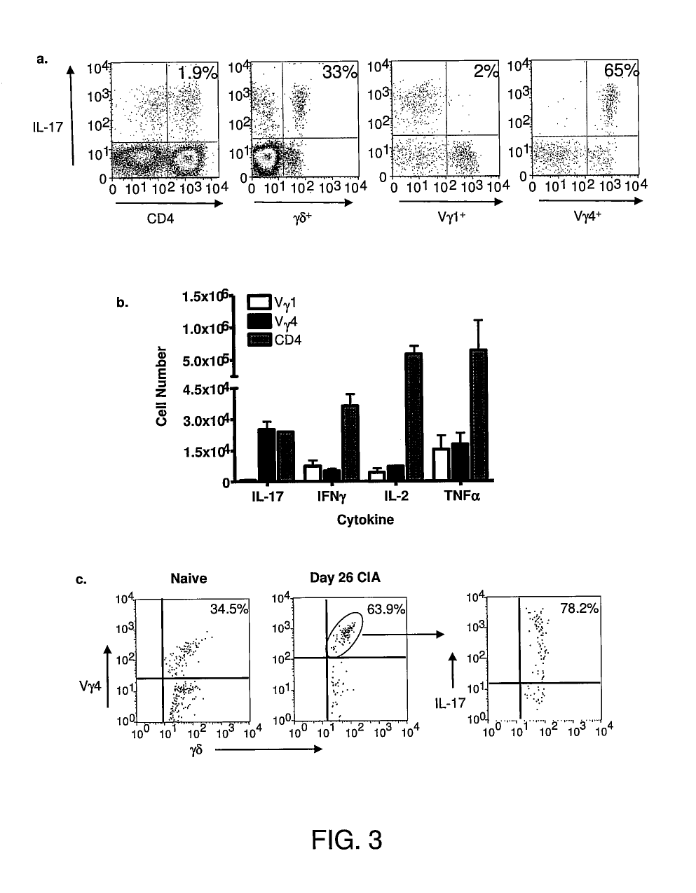

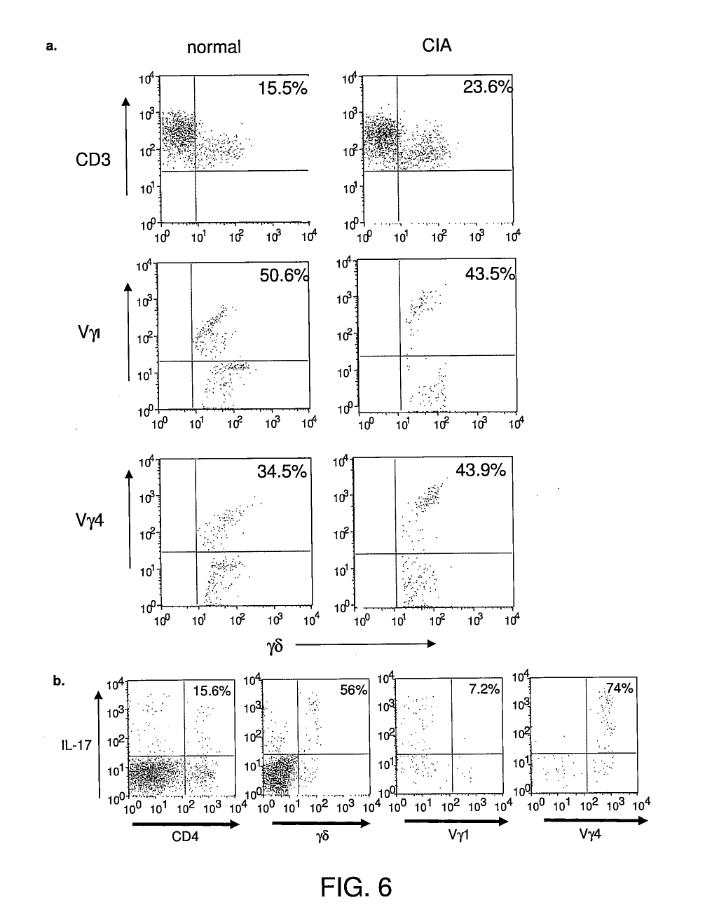

The results described above suggest that Vγ4⁺ cells are pathogenic in CIA. To determine how Vδ4⁺ γδ T cells mediate their effect, the inventors assessed their cytokine potential. Draining lymph nodes were harvested on day 26, when the total number of Vδ4⁺ cells reaches its peak, and intracellular cytokine staining was used to detect IFNγ, IL-2, TNFα, and IL-17 production. In naïve mice, 6% of total γδ T cells, less than 1% of Vγ1⁺ cells, and 20% of Vδ4⁺ cells produced IL-17 (data not shown). However, in CIA mice, 33% of γδ T cells produced IL-17 (FIG. 3a). When the γδ T cell subsets were analyzed, only 2% of Vγ1⁺ cells as compared to 65% of Vγ4⁺ cells produced IL-17 (FIG. 3a). In fact, Vγ4⁺ cells represented over 90% of the total γδ T cells that produced IL-17 in CIA. The fraction (not shown) and number of Vγ1⁺ and Vγ4⁺ cells that produced TNFα, IL-2, and IFNγ were similar (FIG. 3b). Since IL-17 is an inflammatory cytokine produced by activated CD4⁺ αβ T cells (Th17 cells)¹⁷⁻²⁰, the number of CD4⁺ cells and Vγ4⁺ cells that produced IL-17 was also compared. Remarkably, despite its small size, the Vγ4⁺ population contained as many or more IL-17 producers than all CD4⁺ αβ T cells taken together, suggesting that Vγ4⁺ cells are a critical source of IL-17 (FIG. 3b). The cytokine potential of γδ T cells from the joints of normal DBA/1 mice and CIA mice was also characterized. The inventors found a substantial percentage of TCR γδ⁺ cells among T cells in the joints of normal animals (15%) and even more, approximately 23%, in the joints of diseased paws. The percentage of Vγ4⁺ cells was also increased in the diseased joints, while the percentage of Vγ1⁺ cells was decreased (FIG. 6). In addition, a large fraction of Vγ4⁺ cells taken from the joints produced IL-17 at day 26 of the disease process (FIG. 3c).

### Example 4

The following example shows that CIA-elicited Vγ4⁺ cells preferentially express Vδ4.

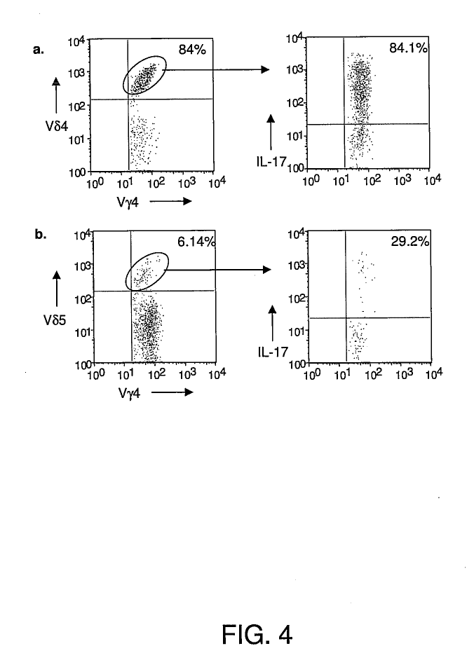

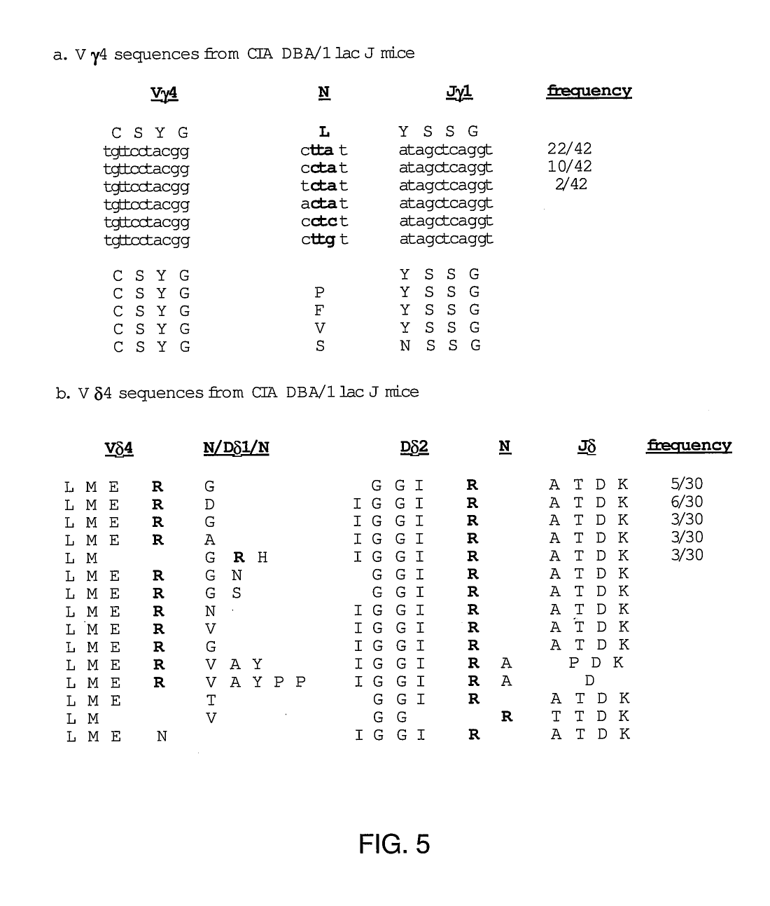

While the function of γδ T cells has been shown to primarily segregate with Vγ chain usage, a recent study by Shin et al. implied that some γδ T cells recognize their ligand primarily through the junctional region of the delta chain²¹. Therefore, the inventors looked at the delta chains co-expressed by CIA-elicited Vγ4⁺ cells. Surprisingly, the inventors discovered that 84% of the CIA-elicited Vγ4⁺ cells co-expressed Vγ4, and that these cells also represented the vast majority of the IL-17 producers (FIG. 4a). In naïve animals, the frequency of Vγ4⁺ cells co-expressing Vδ4⁺ was approximately 20% (data not shown). Of the few Vγ4/Vδ5⁺ cells in the lymph nodes of the CIA mice, a small percentage produced IL-17 (FIG. 4b). Very few Vγ4/Vδ6.3⁺ cells were detected (data not shown). Sequence analysis of day 26 lymph node cDNA revealed a strikingly limited junctional region in the Vδ4 chain, suggesting an antigen-driven clonal response (FIG. 5a). Specifically, 88% of the Vγ4⁺ sequences (37/42) encoded identical CDR3 regions which contained a leucine, encoded by N or P-nucleotides, in the V-J junctional region. Multiple codon triplets were found encoding this leucine, suggesting that this population did not result from a single clonal expansion. Instead, the oligoclonal response may have been driven by specific ligand recognition. The Vδ4 sequences were also limited in variability, with most showing both length conservation and exclusive use of a single Dδ2 reading frame (FIG. 5b). In addition, two conserved arginine codons were found in nearly all sequences, the first encoded by either the 3′ end of the Vδ4 gene or by N-additions, and the second encoded by the 3′ end of the Dδ2 gene. Small groups of identical Vδ4 clones were also evident. In contrast, Vδ4 and Vδ4 sequences from naïve DBA/1 mice were highly variable (Supplementary FIGS. 2 & 3 online). Importantly, no identical Vδ clones were found in the naïve animals.

### REFERENCES

1. Luross, J. A. & Williams, N. A. The genetic and immunopathological processes underlying collagen-induced arthritis. Immunology 103, 407-16 (2001).
2. Gregersen, P. K., Silver, J. & Winchester, R. J. The shared epitope hypothesis. An approach to understanding the molecular genetics of susceptibility to rheumatoid arthritis. Arthritis Rheum. 30, 1205-13. (1987).
3. Wooley, P. H., Whalen, J. D. & Chapdelaine, J. M. Collagen-induced arthritis in mice. VI. Synovial cells from collagen arthritic mice activate autologous lymphocytes in vitro. Cell Immunol. 124, 227-38 (1989).
4. Andersson, E. C. et al. Definition of MHC and T cell receptor contacts in the HLA-DR4 restricted immunodominant epitope in type II collagen and characterization of collagen-induced arthritis in HLA-DR4 and human CD4 transgenic mice. Proc. Natl. Acad. Sci. U.S.A. 95, 7574-9 (1998).
5. Watson, W. C. & Townes, A. S. Genetic susceptibility to murine collagen II autoimmune arthritis. Proposed relationship to the IgG2 autoantibody subclass response, complement C5, major histocompatibility complex (MHC) and non-MHC loci. J. Exp. Med. 162, 1878-91 (1985).
6. Corthay, A., Johansson, A., Vestberg, M. & Holmdahl, R. Collagen-induced arthritis development requires alpha beta T cells but not gamma delta T cells: studies with T cell-deficient (TCR mutant) mice. Int. Immunol. 11, 1065-73. (1999).
7. Peterman, G. M., Spencer, C., Sperling, A. I. & Bluestone, J. A. Role of γδ T cells in murine collagen-induced arthritis. J. Immunol. 151, 6546-6558 (1993).
8. Arai, K. et al. Extrathymic differentiation of resident T cells in the joints of mice with collagen-induced arthritis. J. Immunol. 157, 5170-7 (1996).
9. Holoshitz, J., Koning, F., Coligan, J. E., De Bruyn, J. & Strober, S. Isolation of CD4-CD8⁻ mycobacteria-reactive T lymphocyte clones from rheumatoid arthritis synovial fluid. Nature 339, 226-229 (1989).
10. Kjeldsen-Kragh, J. et al. T γδ cells in juvenile rheumatoid arthritis and rheumatoid arthritis. Scand. J. Immunol. 32, 651-660 (1990).
11. Brennan, F. M. et al. T cells expressing γδ chain receptors in rheumatoid arthritis. J. Autoimmun. 1, 319-326 (1988).
12. Sperling, A. I., Cron, R. Q., Decker, D. C., Stern, D. A. & Bluestone, J. A. Peripheral T cell receptor γδ variable gene repertoire maps to the T cell receptor loci and is influenced by positive selection. J. Immunol. 149, 3200-3207 (1992).
13. Pereira, P., Gerber, D., Huang, S. Y. & Tonegawa, S. Ontogenic development and tissue distribution of Vγ1-expressing γ/δ T lymphocytes in normal mice. J. Exp. Med. 182, 1921-1930 (1995).
14. O'Brien, R. L., Lahn, M., Born, W. K. & Huber, S. A. T cell receptor and function cosegregate in gamma-delta T cell subsets. Chem. Immunol. 79, 1-28 (2001).
15. Steinman, L. A brief history of T(H)17, the first major revision in the T(H)1/T(H)2 hypothesis of T cell-mediated tissue damage. Nat. Med. 13, 139-45 (2007).
16. Tough, D. F. & Sprent, J. Turnover of naive- and memory-phenotype T cells. J. Exp. Med. 179, 1127-1135 (1994).
17. Yao, Z. et al. Human IL-17: a novel cytokine derived from T cells. J. Immunol. 155, 5483-6 (1995).
18. Fossiez, F. et al. T cell interleukin-17 induces stromal cells to produce proinflammatory and hematopoietic cytokines. J. Exp. Med. 183, 2593-603 (1996).
19. Infante-Duarte, C., Horton, H. F., Byrne, M. C. & Kamradt, T. Microbial lipopeptides induce the production of IL-17 in Th cells. J. Immunol. 165, 6107-15 (2000).
20. Spriggs, M. K. Interleukin-17 and its receptor. J. Clin. Immunol. 17, 366-9 (1997).
21. Shin, S. et al. Antigen recognition determinants of gammadelta T cell receptors. Science 308, 252-5 (2005).
22. Arden, B., Clark, S. P., Kabelitz, D. & Mak, T. W. Mouse T-cell receptor variable gene segment families. Immunogenetics 42, 501-530 (1995).
23. O'Brien R, L. et al. gammadelta T-cell receptors: functional correlations. Immunol. Rev. 215, 77-88 (2007).
24. Hahn, Y. S. et al. V gamma 4+ gamma delta T cells regulate airway hyperreactivity to methacholine in ovalbumin-sensitized and challenged mice. J. Immunol. 171, 3170-8 (2003).
25. Hahn, Y. S. et al. Different potentials of gamma delta T cell subsets in regulating airway responsiveness: V gamma 1+ cells, but not V gamma 4+ cells, promote airway hyperreactivity, Th2 cytokines, and airway inflammation. J. Immunol. 172, 2894-902 (2004).
26. Huber, S. A., Graveline, D., Newell, M. K., Born, W. K. & O'Brien, R. L. Vγ 1⁺T cells suppress and Vγ4⁺ T cells promote susceptibility to coxsackievirus B3-induced myocarditis in mice. J. Immunol. 165, 4174-4181 (2000).
27. Huber, S. A., Graveline, D., Born, W. K. & O'Brien, R. L. Cytokine production by Vγamma(+)-T-cell subsets is an important factor determining CD4(+)-Th-cell phenotype and susceptibility of BALB/c mice to coxsackievirus B3-induced myocarditis. J. Virol. 75, 5860-9 (2001).
28. Stark, M. A. et al. Phagocytosis of apoptotic neutrophils regulates granulopoiesis via IL-23 and IL-17. Immunity 22, 285-94 (2005).
29. Lockhart, E., Green, A. M. & Flynn, J. L. IL-17 production is dominated by gammadelta T cells rather than CD4 T cells during Mycobacterium tuberculosis infection. J. Immunol. 177, 4662-9 (2006).
30. Nakae, S., Nambu, A., Sudo, K. & Iwakura, Y. Suppression of immune induction of collagen-induced arthritis in IL-17-deficient mice. J. Immunol. 171, 6173-7 (2003).
31. Lubberts, E. et al. Treatment with a neutralizing anti-murine interleukin-17 antibody after the onset of collagen-induced arthritis reduces joint inflammation, cartilage destruction, and bone erosion. Arthritis Rheum. 50, 650-9 (2004).
32. Heilig, J. S. & Tonegawa, S. T-cell γ gene is allelically but not isotypically excluded and is not required in known functional T-cell subsets. Proc. Natl. Acad. Sci. U.S.A. 84, 8070-8074 (1987).
33. Julius, M. H., Simpson, E. & Herzenberg, L. A. A rapid method for the isolation of functional thymus-derived murine lymphocytes. Eur. J. Immunol. 3, 645-649 (1973).
34. Goodman, T., LeCorre, R. & Lefrancois, L. A T-cell receptor γδ-specific monoclonal antibody detects a Vγ5 region polymorphism. Immunogenetics 35, 65-68 (1992).
35. Dent, A. L. et al. Self-reactive γδ T cells are eliminated in the thymus. Nature 343, 714-719 (1990).
36. Tomonari, K. A rat antibody against a structure functionally related to the mouse T cell receptor/T3 complex. Immunogenetics 28, 455-458 (1988).
37. Bendele, A. M. et al. Combination benefit of treatment with the cytokine inhibitors interleukin-1 receptor antagonist and PEGylated soluble tumor necrosis factor receptor type I in animal models of rheumatoid arthritis. Arthritis Rheum. 43, 2648-59 (2000).
38. Lahn, M. et al. MHC class I-dependent Vγamma4+ pulmonary T cells regulate alpha beta T cell-independent airway responsiveness. Proc. Natl. Acad. Sci. U.S.A. 99, 8850-5 (2002).
39. O'Brien, R. L. et al. Heat shock protein Hsp-60 reactive γδ cells: A large, diversified T lymphocyte subset with highly focused specificity. Proc. Natl. Acad. Sci. U.S.A. 89, 4348-4352 (1992).
40. Pereira, P. et al. Developmentally regulated and lineage-specific rearrangement of T cell receptor Valpha/delta gene segments. Eur. J. Immunol. 30, 1988-97 (2000).
41. Belles, C. et al. Bias in the γδ T cell response to Listeria monocytogenes. Vδ6.3⁺ cells are a major component of the γδ T cell response to Listeria monocytogenes. J. Immunol. 156, 4280-4289 (1996).

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A method to reduce the severity or incidence of a disease or condition associated with the production of interleukin-17 (IL-17), comprising deleting, inactivating or inhibiting γδ T cells in an individual who has or is at risk of developing the disease.

2. The method of claim 1, wherein the disease or condition is an autoimmune disease.

3. The method of claim 2, wherein the autoimmune disease is selected from the group consisting of: rheumatoid arthritis, systemic lupus erythematosus, and multiple sclerosis.

4. The method of claim 3, wherein the autoimmune disease is rheumatoid arthritis.

5. The method of claim 1, wherein the disease or condition is a cancer associated with the production of interleukin-17 (IL-17).

6. The method of claim 5, wherein the cancer is a cancer of a mucosal tissue or organ.

7. The method of claim 5, wherein the cancer is selected from the group consisting of: melanomas, squamous cell carcinomas, breast cancers, head and neck carcinomas, thyroid carcinomas, soft tissue sarcomas, bone sarcomas, testicular cancers, prostatic cancers, pancreatic cancers, ovarian cancers, uterine cancers, cervical cancers, bladder cancers, skin cancers, brain cancers, angiosarcomas, hemangiosarcomas, mast cell tumors, primary hepatic cancers, lung cancers (including non-small cell lung carcinomas), pancreatic cancers, gastrointestinal cancers (including colorectal cancers), renal cell carcinomas, hematopoietic neoplasias and metastatic cancers thereof.

8. The method of claim 5, wherein the cancer is selected from the group consisting of: uterine cancer and colorectal cancer.

9. The method of claim 1, wherein the disease or condition is an inflammatory condition associated with the production of interleukin-17 (IL-17).

10. The method of claim 9, wherein the inflammatory condition is an inflammatory condition of a mucosal organ or tissue.

11. The method of claim 9, wherein the inflammatory condition is not a bacterial or mycobacterial infection.

12. The method of claim 1, comprising selectively deleting, inactivating or inhibiting γδ T cells that produce IL-17 in the individual.

13. The method of claim 1, comprising selectively deleting, inactivating or inhibiting γδ T cells in the individual that produce IL-17 and express activation markers.

14. The method of claim 1, wherein the activation markers include CD44.

15. The method of claim 1, wherein the γδ T cells have reduced expression of CD62L or CD45RB, as compared to γδ T cells in an individual that does not have the autoimmune disease.

16. The method of claim 1, comprising deleting, inactivating or inhibiting a population of γδ T cells in the individual that produce IL-17 and have a T cell receptor comprised of the same Vγ and Vδ combination.

17. The method of claim 1, comprising deleting, inactivating or inhibiting a population of γδ T cells in the individual that produce IL-17 and have a T cell receptor with a highly conserved amino acid motif in the CDR3 region of the TCR-δ chain.

18. The method of claim 1, comprising deleting, inactivating or inhibiting a population of γδ T cells in the individual that produce IL-17 and have a T cell receptor with a highly conserved amino acid motif in the CDR3 region of the TCR-γ chain.

19. The method of claim 1, comprising deleting, inactivating or inhibiting γδ T cells having a T cell receptor comprising Vδ4 or the human equivalent thereof

20. The method of claim 1, comprising deleting, inactivating or inhibiting γδ T cells having a T cell receptor comprising Vδ4 or the human equivalent thereof, and comprising Vδ4 or the human equivalent thereof.

21. The method of claim 1, wherein the γδ T cells are deleted, inactivated or inhibited by selective leukophoresis.

22. The method of claim 1, wherein the γδ T cells are deleted, inactivated or inhibited by administration of an agent that selectively targets γδ T cells.

23. The method of claim 1, wherein the γδ T cells are deleted, inactivated or inhibited by administration of an agent that selectively targets γδ T cells having a specified Vγ and Vδ combination.

24. The method of claim 1, wherein the γδ T cells are deleted, inactivated or inhibited by administration of an agent that selectively targets γδ T cells expressing TCR-Vδ4, or the human equivalent thereof.

25. The method of claim 1, wherein the γδ T cells are deleted, inactivated or inhibited by administration of an agent that selectively targets γδ T cells expressing TCR-Vδ4/Vδ4, or the human equivalent thereof.

26. The method of claim 22, wherein the agent is an antibody or antigen-binding fragment thereof.

27. The method of claim 22, wherein the agent is a soluble γδ T cell receptor identical or equivalent to that expressed by the γδ T cells to be deleted, inactivated or inhibited.

28. A method to reduce the severity or incidence of an autoimmune disease in an individual, comprising deleting, inactivating or inhibiting γδ T cells that produce interleukin-17 (IL-17) in the individual.

29. The method of claim 28, wherein the autoimmune disease is rheumatoid arthritis, systemic lupus erythematosus, or multiple sclerosis.

30. The method of claim 29, comprising selectively deleting, inactivating or inhibiting γδ T cells in the joints of the individual; and wherein the autoimmune disease is rheumatoid arthritis.

31. A method to identify an agent useful for the treatment of a disease or condition associated with the production of interleukin-17 (IL-17), comprising:
a) contacting γδ T cells that produce IL-17 with a putative agent; and
b) selecting a putative agent that deletes or inactivates the γδ T cells of (a) as an agent for the treatment of the disease or condition.

32. The method of claim 31, wherein the γδ T cells of step (a) produce IL-17 and express TCR-Vδ4, or the human equivalent thereof.

33. The method of claim 31, wherein the γδ T cells were obtained or derived from a patient with a disease or condition associated with the production of IL-17.

34. The method of claim 31, wherein the step of selecting comprises selecting a putative agent that inhibits the production of IL-17 by the γδ T cells.

35. The method of claim 31, wherein the γδ T cells express TCR-Vδ4/Vδ4, or the human equivalent thereof.

36. The method of claim 31, wherein the disease is an autoimmune disease.

37. The method of claim 36, wherein the autoimmune disease is rheumatoid arthritis or systemic lupus erythematosus.

38. The method of claim 31, wherein the disease is a cancer.

39. The method of claim 31, wherein the condition is an inflammatory condition.

40. The method of claim 39, wherein the inflammatory condition is associated with a mucosal tissue or organ.
